(19) United States
(12) Patent Application Publication
(10) Pub. No.: US 2024/0166541 A1
(43) Pub. Date: May 23, 2024

(54) METHOD FOR REMOVAL OF NOXIOUS TASTE OR ODOR COMPOUNDS FROM AQUACULTURE SYSTEM BY BIOACTIVE HYDROPHOBIC CARRIERS

(71) Applicant: YISSUM RESEARCH DEVELOPMENT COMPANY OF THE HEBREW UNIVERSITY OF JERUSALEM LTD., Jerusalem (IL)

(72) Inventors: Amos NUSSINOVITCH, Rehovot (IL); Jaap VAN RIJN, Rehovot (IL)

(21) Appl. No.: 18/283,019

(22) PCT Filed: Mar. 20, 2022
(86) PCT No.: PCT/IL2022/050308

§ 371 Date: Sep. 20, 2023

Related U.S. Application Data
(60) Provisional application No. 63/163,891, filed on Mar. 21, 2021.

(51) Int. Cl. C02F 3/10; A01K 63/04; C02F 3/00; C02F 3/08; C02F 3/28; C02F 101/34; C02F 103/20

(52) U.S. Cl. CPC C02F 3/108; A01K 63/04; C02F 3/085; C02F 3/106; C02F 3/2806; C02F 2003/001; C02F 2101/34; C02F 2103/20; C02F 2303/02; C02F 2305/06

(57) ABSTRACT

The present invention provides a method for the removal of noxious taste or odor compounds (TOCs) from water of an aquaculture system, the method comprising contacting the water with a carrier comprising: a hydrophobic agent being configured to adsorb said TOCs; and a hydrocolloid, wherein the carrier is adapted for the colonization of microorganisms which are capable of degrading said TOCs. Further provided is a recirculating aquaculture system (RAS) for maintaining aquaculture species, comprising, inter alia, a reactor comprising a plurality of carriers comprising a hydrophobic agent configured to adsorb TOCs, and a hydrocolloid.

### FIELD OF THE INVENTION

The present invention is directed to the method for removal of noxious taste or odor compounds from an aquaculture system by bioactive hydrophobic carriers comprising a hydrophobic agent and a hydrocolloid, and to a recirculating aquaculture system comprising said carriers.

### BACKGROUND OF THE INVENTION

Aquaculture, with an estimated value of 160 billion US$, is one of the fastest growing food production sectors and currently accounts for nearly half of the world's fish supply. Given the stagnant global landings by captured fisheries and the rapidly expanding global fish demand, it is clear that aquaculture will continue to fill the growing supply-demand gap in aquatic food products.

The global expansion of the aquaculture sector depends, among other factors, on the development of sustainable practices whereby land and water resources are exploited in an efficient manner. With the development of such culture systems in which fish are grown at high densities, the problem of so called “off-flavored” fish has increased. This problem is caused mainly by two noxious taste and odor terpenoid compounds, i.e., geosmin and 2-methylisoborneol (also termed herein “MIB”), which are produced as secondary metabolites by several microorganisms, such as, e.g., cyanobacteria (also termed “blue-green algae”) and actinobacteria. The production of geosmin and MIB takes place in both conventional and more intensive aquaculture facilities. When released into aquaculture water bodies, said hydrophobic chemicals accumulate in the fatty tissues of fish and render the fish unmarketable due to their distinct muddy (or earthy) taste and odor. Both compounds are highly potent and may produce off-flavored fish when present at very low concentrations in the culture water (e.g., about 20 ng/L). In contaminated aquaculture facilities, geosmin and MIB are usually present within the concentration range of 100-500 ng/L. The problem of off-flavor imposes heavy financial constraints on the aquaculture industry.

Several methods for removal of geosmin and MIB from water are presently employed. One of the methods is based on geosmin and MIB adsorption on hydrophobic media. In the drinking water industry, where off flavor-tainted water has caused severe financial burdens as a result of consumer complaints, activated carbon is widely used for this purpose. Although effective in treating water with low organic matter concentrations, this method is less efficient in organic-rich aquaculture systems where, like in the treatment of other organic-rich water bodies, filters may become ineffective due to clogging and activated carbon must be frequently replaced.

U.S. Pat. No. 4,398,937 is directed to a method of selectively controlling the growth of blue-green algae and yellow-green algae, the method comprising adding from about 0.01 to about 100 ppm of a fatty acid compound selected from the group consisting of 9, 10-dihydroxystearic acid, Dodecanoic acid, Linoleic acid and Ricinoleic acid to the aqueous medium.

U.S. Pat. No. 6,902,675 is directed to a method for the extraction of terpenoids in aquatic environments by using a hydrophobic absorbent, thus reducing or eliminating off-flavor in water and aquaculture products which is caused by cyanobacteria-produced terpenoids, the method comprising the steps of: extracting one or more said terpenoids from said water with a water-insoluble hydrophobic sorbent with the formation of a terpenoid-depleted water phase and a terpenoid-enriched sorbent phase and leaving the solvent phase in said water to undergo degradation or evaporation. The hydrophobic absorbent can include liquid or solid compounds that absorb terpenoids and thereby remove such terpenoids from water and aquaculture products, including, inter alia, naturally occurring polymers, synthetic polymers, waxes and oils. The hydrophobic absorbent can be placed in a porous container, such as, filter, bag, trap, or screened cage, which allows confinement, collection, recovery, reuse, disposal, or regeneration of the hydrophobic absorbent.

However, the addition of liquid oil to an aquaculture system can lead to rapid system failure, as recirculating aquaculture systems (RASs) are not designed to treat or remove oily substances. When present in RAS, liquid oil can severely damage crucial biofilms and other water treatment units. Likewise, when contacting water current, wax will rapidly dissociate and will eventually break down into small, oily particles that will damage the system's operation. Synthetic polymers disclosed in U.S. Pat. No. 6,902,675 have not been shown to efficiently remove geosmin and/or MIB from aquaculture water. In addition, the method disclosed in U.S. Pat. No. 6,902,675 is based on a mere separation of the off-flavor compounds from the culture water and does not provide degradation of the off-flavor compounds. Hence, a frequent replacement/disposal of the absorbance matrix is required.

U.S. Pat. No. 6,063,287 discloses a process for the removal of algae-associated odorant from fresh water by contacting such water with cyclodextrin and recovering the water so contacted. This approach requires the use of cyclodextrins that are relatively expensive and which need to be separated from the water following odorant adsorption and regenerated in a separate process.

JP 2019018179 is directed to a method for removing a musty odor substance comprising at least one of geosmin and 2-methylisoborneol contained in water to be treated, comprising, inter alia, mixing the water to be treated with a cyclic compound capable of forming an inclusion compound with at least one of geosmin and 2-methylisoborneol and contacting the water mixed with the cyclic compound with an ion exchanger. The cyclic compound can be selected from cyclodextrin, calixarene, and derivatives thereof.

Oxidation with strong oxidizing agents such as ozone and chlorine is an additional method for removal of said noxious taste or odor terpenoid compounds. These oxidation methods, mainly used in the drinking water industry, are less appropriate for aquaculture systems. In their study on ozonation in recirculating aquaculture systems (RAS), Schrader et al. (Schrader, K. K., et al., 2010, Aquacultural Engineering 43: 46-50) concluded that ozone doses, routinely applied to improve water quality parameters in RAS, were not sufficiently high for removal of geosmin and MIB from the culture water. Use of higher doses, besides being costly, would increase the risk of a breakthrough of ozone in the culture water and thereby endanger the fish and potentially damage critical bacterial biofilms within the systems' filtration units.

The currently known methods of reducing aquaculture system contamination by noxious taste or odor compounds (TOCs) are either expensive, toxic or inefficient. The major operational disadvantage of the use of hydrophobic agents, as well as its economic burden, is the need to regenerate the hydrophobic agent following the removal of the contaminants from the aquaculture system. In the absence of effective means for prevention of geosmin and MIB accumulation in RAS, depuration of marketable fish is currently the most common abatement method. Purging is a laborious, time consuming and expensive process.

There remains, therefore, an unmet need for the commercially cost-effective, environmentally friendly, efficient and easily-implementable methods for geosmin and MIB removal from aquaculture systems.

### SUMMARY OF THE INVENTION

The present invention provides a method for the removal of noxious taste or odor compounds (TOCs) from aquaculture systems. The removal method is based on the adsorption and biodegradation of said compounds, which are effected by the use of a single bioactive carrier. Said bioactive carrier has a uniquely designed composition and/or structure, which account for its ability to adsorb noxious taste or odor terpenoid compounds and to induce development of a population of microorganisms, which degrade or transform said adsorbed compounds. The bioactive carrier includes a combination of a hydrophobic agent, which is liquid, semi-solid or solid at the operating temperatures of the aquaculture system, and a hydrocolloid. Preferably, the hydrocolloid is configured to support or entrap the hydrophobic agent and the hydrophobic agent is configured to adsorb the TOCs. The hydrophobic agent can be dispersed within the hydrocolloid network or the hydrocolloid can be disposed within the solid or semi-solid hydrophobic agent. One of the exemplary embodiments includes a carrier being in the form of an isotropic particle in which the hydrophobic agent is homogeneously dispersed within the hydrocolloid gel network, thereby maximizing the surface area, which is available for the colonization of the terpenoid-degrading bacteria and the bulk, which is configured to adsorb the off-flavor terpenoid compounds. Another set of exemplary embodiments relates to a carrier comprising a solid or semi-solid agent in which a powdered hydrocolloid is dispersed.

The physical-biological approach of the current TOCs removal method was found to be effective, environmentally safe, technologically uncomplicated and cost-efficient. The present invention is based in part on an unexpected finding that a priori provision of terpenoid-degrading microorganisms' population within the bioactive carrier or on its surface was not essential for the efficient removal of geosmin and MIB, when the bioactive carrier was formulated according to the principles of the present invention. Unexpectedly, endemic population of microorganisms had developed spontaneously on the carrier surface of bacteria-less carriers upon the contact of the carrier with contaminated water from the aquaculture system and the structure of said microorganisms' population (i.e., the types of the microorganisms and their relative abundance) was found to be particularly suitable for the degradation of TOCs, such as geosmin and MIB. Without wishing to being bound by theory or mechanism of action, it is contemplated that the bioactive carrier of the present invention is highly selective towards colonization of endemic terpenoid off-flavor-degrading bacteria on the carrier surface. The use of endemic bacteria, which are frequently found in RAS, is beneficial as compared to the use of bacteria isolated from a foreign culture and preliminarily formed within the carrier, as endemic bacteria are specifically adapted for the prevailing environmental conditions in the specific aquaculture system to be treated. Accordingly, the bioactive carrier can be prepared by a simple chemical manufacturing process, which does not require a time-consuming, expensive and technologically challenging step of growing biological species. Advantageously, the method of the present invention provides combined adsorption and degradation of noxious TOCs found in an aquaculture system without the use of toxic bioactive chemical agents or carrier regeneration process or system (e.g., ion exchange column). The treated water can be reintroduced to the aquaculture system and the bioactive carriers can be reused without any further desorption or cleaning procedures.

In one aspect, the present invention provides a method for the removal of noxious taste or odor compounds (TOCs) from water of an aquaculture system, the method comprising contacting the water with a carrier comprising: a hydrophobic agent configured to adsorb said noxious TOCs; and a hydrocolloid, wherein the carrier is adapted for the colonization of microorganisms which are capable of degrading said TOCs and wherein said TOCs comprise a terpenoid.

According to some embodiments, the hydrocolloid is configured to entrap the hydrophobic agent. According to some embodiments, the hydrocolloid is dispersed within the hydrophobic agent.

According to some embodiments, the terpenoid is selected from trans-1,10-dimethyl-trans-9-decalol (geosmin) and 2-methylisoborneol (MIB). According to further embodiments, the terpenoid is present in the water in a concentration ranging from about 10 to about 1000 ng/L.

The hydrophobic agent can be selected from the group consisting of an oil, wax, fatty acid, fatty alcohol, and combinations thereof. Each possibility represents a separate embodiment of the invention.

In certain embodiments, the hydrophobic agent comprises the oil. In further embodiments, the oil is selected from the group consisting of soybean oil, coconut oil, cottonseed oil, peanut oil, rapeseed oil, canola oil, safflower oil, peanut oil, walnut oil, sesame oil, olive oil, linseed oil, evening primrose oil, sea buckthorn oil, palm oil, sunflower oil, corn oil, jojoba oil, marrow oil, grapeseed oil, hazelnut oil, apricot oil, macadamia oil, almond oil, castor oil, acai berry oil, apricot oil, avocado oil, baobab oil, black cumin oil, blackcurrant seed oil, blueberry seed oil, borage oil, camelina oil, cherry kernel oil, chia seed oil, cranberry seed oil, hemp seed oil, macadamia nut oil, marula oil, neem oil, oat oil, argan oil, pomegranate seed oil, peach kernel oil, plum kernel oil, barbary fig seed oil, raspberry seed oil, rice bran oil, rosehip seed oil, tamanu oil, wheatgerm oil, and combinations thereof. Each possibility represents a separate embodiment of the invention. In some exemplary embodiments, the hydrophobic agent comprises soybean oil. In additional exemplary embodiments, the hydrophobic agent comprises sunflower oil.

In certain embodiments, the hydrophobic agent comprises the wax. The wax can be selected from the group consisting of beeswax, lanolin, hard tallow, Japan wax, castor wax, sugar cane wax, candelilla wax, bayberry wax, cocoa butter, illipe butter, ceresin, petrolatum, paraffin, and combinations thereof. Each possibility represents a separate embodiment of the invention. In some exemplary embodiments, the hydrophobic agent comprises beeswax.

In certain embodiments, the hydrophobic agent comprises the fatty alcohol. The fatty alcohol can be selected from the group consisting of stearyl alcohol, cetyl alcohol, lanolin alcohol, 2-octyldodecanol, and combinations thereof. Each possibility represents a separate embodiment of the invention.

In certain embodiments, the hydrophobic agent comprises the fatty acid. The fatty acid can be selected from the group consisting of oleic acid, stearic acid, palmitic acid, lauric acid, myristic acid, behenic acid, isostearic acid, and combinations thereof. Each possibility represents a separate embodiment of the invention.

According to some embodiments, the hydrophobic agent comprises at least one of soybean oil, sunflower oil, and beeswax.

The hydrophobic agent can be present in the carrier in a weight percent ranging from about 10% to about 40% out of the total wet weight of the carrier.

In certain embodiments, the carrier comprises soybean oil, which is present in a weight percent of about 30% out of the total wet weight of the carrier.

According to some embodiments, the hydrophobic agent is present in the carrier in a weight percent ranging from about 88% to about 99.5% out of the total dry weight of the carrier.

According to some embodiments, the hydrocolloid is selected from the group consisting of a natural hydrocolloid, a synthetic or semisynthetic hydrocolloid, and any combination thereof. The natural hydrocolloid can be selected from the group consisting of alginate, agar, agarose, gelatin, low methoxy pectin (LMP), chitosan, gellan, carrageenan, locust bean gum (LBG), guar gum, carrageenan, arabinoxylan, cellulose, curdlan, furcellaran, gellan, β-glucan, starch, modified starch, gum arabic, gum tragacanth, tamarind gum, fenugreek gum, cassia gum, tara gum, xanthan, pullulan, egg protein, vegetable protein, dairy protein, and combinations thereof. Each possibility represents a separate embodiment of the invention. In some exemplary embodiments, the hydrocolloid comprises alginate.

The synthetic or semisynthetic hydrocolloid can be selected from the group consisting of methylcellulose, carboxymethylcellulose, polyacrylates, high molecular weight polyethylene glycols and polypropylene glycols, polyethylene oxides, polyacrylic acid polymers, poly vinyl alcohols, and combinations thereof. Each possibility represents a separate embodiment of the invention.

The hydrocolloid can be present in the carrier in a weight percent ranging from about 0.1% to about 5% out of the total wet weight of the carrier. In some embodiments, the hydrocolloid is present in the carrier in a weight percent ranging from about 0.01% to about 2% out of the total dry weight of the carrier.

According to some embodiments, the carrier further comprises a cross-linking agent. The cross-linking agent can be selected from the group consisting of calcium ion, magnesium ion, potassium ion, barium ion, aluminum ion, copper ion, lead ion, strontium ion, chitosan, poly(L-lysine), polyethyleneimine (PEI), and combinations thereof. Each possibility represents a separate embodiment of the invention. In some embodiments, the cross-linking agent is present in the carrier in a weight percent ranging from about 0.1% to about 5% out of the total wet weight of the carrier.

According to some embodiments, the carrier further comprises an emulsifier. The emulsifier can be selected from the group consisting of ethoxylated sorbitan esters of fatty acids, succinylated monoglycerides, sucrose esters of fatty acids, sucroglycerides, polyglycerol esters of fatty acids, polyglycerol polyricinoleate, propane-1,2-diol esters of fatty acids, propylene glycol esters of fatty acids, lactylated fatty acid esters of glycerol and propane-1, thermally oxidized soya bean oil interacted with mono- and diglycerides of fatty acids, dioctyl sodium sulfosuccinate, sodium stearoyl-2-lactylate, calcium stearoyl-2-lactylate, stearyl tartrate, stearyl citrate, sodium stearoyl fumarate, calcium stearoyl fumarate, sodium lauryl sulfate, ethoxylated mono- and di-glycerides, methyl glucoside-coconut oil ester, propane-1,2-diol, sorbitan monostearate, sorbitan tristearate, sorbitan monolaurate, sorbitan monooleate, sorbitan monopalmitate, sorbitan trioleate, polyoxypropylene-polyoxyethylene polymers, partial polyglycerol esters of polycondensed fatty acids of castor oil, stigmasterol-rich plant sterols, and combinations thereof. Each possibility represents a separate embodiment of the invention. The emulsifier can be present in the carrier in a weight percent ranging from about 0.001% to about 1% out of the total wet weight of the carrier.

In some embodiments, the carrier further comprises from about 50% (w/w) to about 99% (w/w) water out of the total wet weight of the carrier.

According to some embodiments, the carrier further comprises a filler selected from bentonite, kaolin, and any combination thereof.

In certain embodiments, the carrier comprises: from about 10% (w/w) to about 40% (w/w) hydrophobic agent; from about 0.1% (w/w) to about 5% (w/w) hydrocolloid; from about 0.1% (w/w) to about 5% (w/w) cross-linking agent; from about 0.001% (w/w) to about 1% (w/w) emulsifier; and from about 50% (w/w) to about 95% (w/w) water, out of the total wet weight of the carrier. According to further embodiments, the hydrophobic agent comprises soybean oil, the hydrocolloid comprises alginate and the cross-linking agent comprises calcium ions.

In certain embodiments, the carrier is in a form of a particle in which the hydrophobic agent is homogeneously mixed with the hydrocolloid. In further embodiments, the particle has a particle size ranging from about 100 μm to about 10 cm. The particle can further comprise a water-permeable shell comprising a hydrocolloid.

According to some embodiments, the carrier comprises from about 88% (w/w) to about 99.5% (w/w) of the hydrophobic agent; from about 0.1% (w/w) to about 2% (w/w) of the hydrocolloid; and from about 0.1% (w/w) to about 10% (w/w) of the filler, out of the total dry weight of the carrier. According to further embodiments, the hydrophobic agent comprises a combination of sunflower oil and beeswax. the hydrocolloid comprises alginate and the filler comprises bentonite. According to some embodiments, the microorganisms, which are capable of degrading the TOCs, are endemic to the aquaculture system.

According to some embodiments, the aquaculture system is selected from the group consisting of a recirculating aquaculture system (RAS), a raceway fish farm, multi-trophic aquaculture system, aquaponics system, an aquaculture species pond, an aquaculture species pool, an aquaculture species container, an aquaculture species tank, a live aquaculture species transportation apparatus, and an aquaculture depuration system. In further embodiments, the aquaculture system comprises at least one specie selected from the group consisting of fish, shrimp, prawns, mussels, oysters, crab, lobster, scallop, conch, eel and combinations thereof. Each possibility represents a separate embodiment of the invention.

According to some embodiments, the carrier is in a form of a particle and the method comprises contacting the water with a plurality of said particles which are contained within a receptacle within the aquaculture system. The receptacle can be selected from a mesh bag, a stacked packed column, a reactor or any combination thereof.

According to some embodiments, said contacting comprises extracting a portion of water from an aquaculture species reservoir of the aquaculture system, contacting said portion of water with the plurality of particles and, optionally, reintroducing said portion of water to the reservoir. In certain embodiments, the portion of water is passed through the receptacle in a vertical, horizontal or circular fashion. In some exemplary embodiments, the receptacle comprises a stacked packed column or reactor and hydraulic retention time in said receptacle ranges from about 0.1 to about 20 hours.

According to some embodiments, said contacting comprises a preliminary contacting sub-step being performed for at least about 2 weeks, to colonize the microorganisms on the carrier surface. In further embodiments, said preliminary contacting sub-step comprises passing a sample of the water from the aquaculture system through the receptacle comprising the plurality of said particles.

In another aspect there is provided a recirculating aquaculture system (RAS) for maintaining aquaculture species, comprising: a reservoir holding water and aquaculture species; at least one of a solids removing filter, mechanical filter, and biological filter; and a plurality of carriers comprising a hydrophobic agent configured to adsorb noxious taste or order compounds (TOCs), and a hydrocolloid, wherein said carriers are adapted for the colonization of microorganisms which are capable of degrading or transforming said TOCs.

According to some embodiments, the RAS further comprises a reactor being in fluid flow connection with the reservoir and means for directing flow of a portion of the water from the reservoir to the reactor, wherein the reactor comprises the plurality of carriers.

According to some embodiments, the hydrocolloid is configured to entrap the hydrophobic agent. According to some embodiments, the hydrocolloid is dispersed within the hydrophobic agent.

The hydrophobic agent can be selected from the group consisting of an oil, wax, fatty acid, fatty alcohol, and combinations thereof. Each possibility represents a separate embodiment of the invention.

According to some embodiments, the oil is selected from the group consisting of soybean oil, coconut oil, cottonseed oil, peanut oil, rapeseed oil, canola oil, safflower oil, peanut oil, walnut oil, sesame oil, olive oil, linseed oil, evening primrose oil, sea buckthorn oil, palm oil, sunflower oil, corn oil, jojoba oil, marrow oil, grapeseed oil, hazelnut oil, apricot oil, macadamia oil, almond oil, castor oil, acai berry oil, apricot oil, avocado oil, baobab oil, black cumin oil, blackcurrant seed oil, blueberry seed oil, borage oil, camelina oil, cherry kernel oil, chia seed oil, cranberry seed oil, hemp seed oil, macadamia nut oil, marula oil, neem oil, oat oil, argan oil, pomegranate seed oil, peach kernel oil, plum kernel oil, barbary fig seed oil, raspberry seed oil, rice bran oil, rosehip seed oil, tamanu oil, wheatgerm oil, and combinations thereof. Each possibility represents a separate embodiment of the invention.

According to some embodiments, the wax is selected from the group consisting of beeswax, lanolin, hard tallow, Japan wax, castor wax, sugar cane wax, candelilla wax, bayberry wax, cocoa butter, illipe butter, ceresin, petrolatum, paraffin, and combinations thereof. Each possibility represents a separate embodiment of the invention.

According to some embodiments, the fatty acid is selected from the group consisting of oleic acid, stearic acid, palmitic acid, lauric acid, myristic acid, behenic acid, isostearic acid, and combinations thereof. Each possibility represents a separate embodiment of the invention.

The hydrophobic agent can be present in the carrier in a weight percent ranging from about 10% to about 40% out of the total wet weight of the carrier or from about 88% to about 99.5% out of the total dry weight of the carrier.

According to some embodiments, the hydrocolloid comprises a natural hydrocolloid selected from the group consisting of alginate, agar, agarose, gelatin, low methoxy pectin (LMP), chitosan, gellan, carrageenan, locust bean gum (LBG), guar gum, carrageenan, arabinoxylan, cellulose, curdlan, furcellaran, gellan, β-glucan, starch, modified starch, gum arabic, gum tragacanth, tamarind gum, fenugreek gum, cassia gum, tara gum, xanthan, pullulan, egg protein, vegetable protein, dairy protein, and combinations thereof. Each possibility represents a separate embodiment of the invention.

According to some embodiments, the hydrocolloid comprises a synthetic or semisynthetic hydrocolloid selected from the group consisting of methylcellulose, carboxymethylcellulose, polyacrylates, high molecular weight polyethylene glycols and polypropylene glycols, polyethylene oxides, polyacrylic acid polymers, poly vinyl alcohols, and combinations thereof. Each possibility represents a separate embodiment of the invention.

The hydrocolloid can be present in the carrier in a weight percent ranging from about 0.1% to about 5% out of the total wet weight of the carrier or from about 0.01% to about 2% out of the total dry weight of the carrier.

According to some embodiments, the plurality of carriers further comprise a cross-linking agent selected from the group consisting of calcium ion, magnesium ion, sodium ion, potassium ion, barium ion, aluminum ion, copper ion, lead ion, strontium ion, chitosan, poly(L-lysine), polyethyleneimine (PEI), and combinations thereof. Each possibility represents a separate embodiment of the invention. The cross-linking agent can be present in the carrier in a weight percent ranging from about 01% to about 5% out of the total wet weight of the carrier.

According to some embodiments, the plurality of carriers further comprise an emulsifier, selected from the group consisting of ethoxylated sorbitan esters of fatty acids, succinylated monoglycerides, sucrose esters of fatty acids, sucroglycerides, polyglycerol esters of fatty acids, polyglycerol polyricinoleate, propane-1,2-diol esters of fatty acids, propylene glycol esters of fatty acids, lactylated fatty acid esters of glycerol and propane-1, thermally oxidized soya bean oil interacted with mono- and diglycerides of fatty acids, dioctyl sodium sulfosuccinate, sodium stearoyl-2-lactylate, calcium stearoyl-2-lactylate, stearyl tartrate, stearyl citrate, sodium stearoyl fumarate, calcium stearoyl fumarate, sodium lauryl sulfate, ethoxylated mono- and di-glycerides, methyl glucoside-coconut oil ester, propane-1,2-diol, sorbitan monostearate, sorbitan tristearate, sorbitan monolaurate, sorbitan monooleate, sorbitan monopalmitate, sorbitan trioleate, polyoxypropylene-polyoxyethylene polymers, partial polyglycerol esters of polycondensed fatty acids of castor oil, stigmasterol-rich plant sterols, and combinations thereof. Each possibility represents a separate embodiment of the invention. The emulsifier can be present in the carrier in a weight percent ranging from about 0.001% to about 1% out of the total wet weight of the carrier.

In certain embodiments, the plurality of carriers comprises from about 10% (w/w) to about 40% (w/w) hydrophobic agent; from about 0.1% (w/w) to about 5% (w/w) hydrocolloid; from about 0.1% (w/w) to about 5% (w/w) cross-linking agent; from about 0.001% (w/w) to about 1% (w/w) emulsifier; and from about 50% (w/w) to about 95% (w/w) water, out of the total wet weight of the carrier.

The reactor in the RAS can be selected from the group consisting of a packed bed reactor, fixed bed reactor, moving bed reactor, rotating bed reactor, and fluidized bed reactor. In some embodiments, the volume of the reactor ranges from about 0.5 L to about 50,000 L. In further embodiments, the mass of the carriers within the reactor ranges from about 0.1 to about 10,000 kg.

The reservoir in the RAS can be selected from the group consisting of a tank, basin, and pond.

According to some embodiments, the reservoir, at least one of the solids removal filter and mechanical filter, the biological filter, and the reactor with the plurality of carriers are fluidly connected, such that a portion of the water flows from the reservoir to the solids removal filter and/or the mechanical filter; from the solids removal filter and/or mechanical filter to the biological filter; from the biological filter to the reactor with the plurality of carriers; and from the reactor back to the reservoir, or wherein the portion of the water flows from the reservoir to the solids removal filter and/or the mechanical filter; from the solids removal filter and/or mechanical filter to the reactor with the plurality of carriers; from the reactor with the plurality of carriers to the biological filter; and from the biological filter back to the reservoir.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIG. 1A: Graph representing geosmin levels in water prior to the incubation with oil-hydrocolloid carriers (left columns) and after 24 hours of incubation (right columns), wherein the carrier contains 0, 10, 20, 30 and 40% oil (w/w).

FIG. 1B: Graph representing MIB levels in water prior to the incubation with oil-hydrocolloid carrier (left columns) and after 24 hours of incubation (right columns), wherein the carrier contains 0, 10, 20, 30 and 40% oil (w/w).

FIG. 2: Graph representing geosmin (GSM) and MIB levels before (left columns) and after 24 hours of incubation (right columns) in either double distilled water (DDW) or water from a recirculating aquaculture system with 30% (w/w) oil-containing carriers. For control, samples without carriers, incubated under identical conditions, were used (middle columns).

FIG. 3A: Graph representing geosmin levels in water prior to the incubation with 30% (w/w) oil-containing carriers, after 24 hours of incubation, and after 48 hours of incubation, wherein the carriers were first incubated in medium containing crude sludge from a recirculating aquaculture system, known to contain geosmin and MIB degrading bacteria (middle columns). Control trials included carriers, which were not incubated in medium containing crude sludge (right columns) and water samples, which were not incubated with oil-hydrocolloid carriers (left columns).

FIG. 3B: Graph representing MIB levels in water prior to the incubation with 30% (w/w) oil-containing carriers, after 24 hours of incubation, and after 48 hours of incubation, wherein the carriers were first incubated in medium containing crude sludge from a recirculating aquaculture system, known to contain geosmin and MIB degrading bacteria (middle columns). Control trials included carriers, which were not incubated in medium containing crude sludge (right columns) and water samples, which were not incubated with oil-hydrocolloid carriers (left columns).

FIG. 4: Graph representing bacterial count of the carriers following operation in a upflow reactor (squares) and a moving bed reactor (diamonds), in which water from an aquaculture system is circulated.

FIG. 5A: Graph representing ratio of effluent and influent concentration of geosmin during operation of upflow columns packed with either oil-containing hydrocolloid carriers (diamonds) or control hydrocolloid carriers, which do not contain oil (squares) at 10 h HRT, wherein data represent average values of duplicate samples.

FIG. 5B: Graph representing ratio of effluent and influent concentration of MIB during operation of upflow columns packed with either oil-containing hydrocolloid carriers (diamonds) or control hydrocolloid carriers, which do not contain oil (squares) at 10 h HRT, wherein data represent average values of duplicate samples.

FIG. 5C: Graph representing influent (diamonds) and effluent concentration of nitrate during operation of upflow columns packed with either oil-containing hydrocolloid carrier (squares) or control hydrocolloid carriers, which do not contain oil (triangles) at 10 h HRT, wherein data represent average values of duplicate samples.

FIG. 5D: Graph representing influent (diamonds) and effluent pH during operation of upflow columns packed with either oil-containing hydrocolloid carriers (squares) or control hydrocolloid carriers, which do not contain oil (triangles) at 10 h HRT, wherein data represent average values of duplicate samples.

FIG. 5E: Graph representing ratio of effluent and influent concentration of geosmin during operation of upflow columns packed with either oil-containing hydrocolloid carriers (diamonds) or control hydrocolloid carriers, which do not contain oil (squares) at 5 h HRT, wherein data represent average values of 3 samples.

FIG. 6A: Removal of MIB from RAS water by three up-flow columns packed with carriers with the following compositions: alginate-oil, alginate-oil-wax, and alginate-oil-wax-bentonite carriers, and a control column operated under similar conditions, but without carriers.

FIG. 6B: Removal of geosmin from RAS water by three up-flow columns packed with carriers with the following compositions: alginate-oil, alginate-oil-wax, and alginate-oil-wax-bentonite carriers, and a control column operated under similar conditions, but without carriers.

### DETAILED DESCRIPTION OF THE INVENTION

The present invention provides a method for the removal of noxious TOCs from aquaculture systems. The removal method is based on the adsorption and biodegradation of said compounds, and involves the use of a bioactive carrier, comprising a hydrophobic agent and a hydrocolloid. In some embodiments, the hydrocolloid is configured to hold or entrap the hydrophobic agent, which, in turn, is configured to adsorb the TOCs. The surface of the bioactive carrier, which, in some embodiments, has a particulate form, is adapted for the colonization of the microorganisms, which are capable of degrading the adsorbed noxious TOCs. In some embodiments, the hydrocolloid is disposed within the hydrophobic agent.

This physical-biological approach to the off-flavor compounds removal from the aquaculture systems was found to be effective, environmentally safe, technologically uncomplicated and cost-efficient. Unexpectedly, when the bioactive carrier was contacted with water from a RAS, endemic population of microorganisms had developed spontaneously on the carrier surface. Accordingly, the manufacturing of the bioactive carrier does not necessitate growing and maintaining biological species within or upon the carrier, and a population of suitable bacteria can be formed simply by contacting the carrier with the water to be treated. Advantageously, such bioactive carrier affords for a single-step adsorption and degradation process of noxious TOCs found in aquaculture systems.

Further provided is a filter comprising a plurality of said bioactive carriers and a recirculating aquaculture system employing said bioactive carriers for the removal of noxious TOCs.

According to one aspect of the invention, there is provided a method for the removal of noxious taste or odor compounds (TOCs) from water of an aquaculture system, the method comprising contacting the water with a carrier comprising: (a) a hydrophobic agent configured to adsorb said TOCs; and (b) a hydrocolloid, wherein said carrier is adapted for the colonization of microorganisms which are capable of degrading or transforming said TOCs.

In another aspect, there is provided a recirculating aquaculture system (RAS) for maintaining aquaculture species, comprising: a reservoir holding water and aquaculture species; at least one of a solids removing filter, mechanical filter, and biological filter; and a plurality of carriers comprising a hydrophobic agent configured to adsorb noxious taste or order compounds (TOCs), and a hydrocolloid, wherein the carriers are adapted for the colonization of microorganisms which are capable of degrading or transforming said TOCs.

In yet another aspect, there is provided a bioactive filter comprising a plurality of carriers comprising a hydrophobic agent configured to adsorb noxious taste or order compounds (TOCs), and a hydrocolloid, wherein the carriers are adapted for the colonization of microorganisms which are capable of degrading or transforming said TOCs, wherein said bioactive filter is for use in the removal of noxious taste or odor compounds (TOCs) from water of an aquaculture system.

The term “water”, as used herein, refers to an aqueous medium found in an aquaculture species reservoir of the aquaculture system, which may include, in addition to water, various chemicals, such as, but not limited to, nitrogen-containing compounds (e.g., ammonia, ammonium, nitrate, and nitrite), salts, organic compounds (e.g., terpenoids), as well as biological substances (e.g., bacteria). Water may be seawater, brackish water or fresh water. Salinity of the seawater can range from about 3.1% to about 3.8%.

Noxious TOCs, which are also termed herein “off-flavor compounds”, are hydrophobic terpenoids produced by cyanobacteria (commonly known as blue-green algae) and/or actinobacteria and when absorbed by fish, result in undesired off-flavor and odor. Geosmin (trans-1,10-dimethyl-trans-9-decalol) and 2-methylisoborneol (MIB) are two of the known hydrophobic terpenoids produced by cyanobacteria and/or actinobacteria. Other hydrophobic terpenoids may be produced by blue-green algae, either directly as metabolites or as secondary products from metabolites including 2-isopropyl-2-methyloxypyrazine. Accordingly, in some embodiments, said TOCs comprise a terpenoid. According to further embodiments, said terpenoid is produced by cyanobacteria, such as, but not limited to, Oscillatoria species and/or actinobacteria such as, but not limited to, Streptomyces sp. According to still further embodiments, said terpenoid is selected from geosmin, 2-methylisoborneol, 2-isopropyl-2-methyloxypyrazine, and analogs or derivatives thereof. According to certain embodiments, said terpenoid is selected from geosmin and MIB.

The terms “analogue” or “derivative”, as used herein, relate to a chemical molecule that is similar to another chemical substance in structure and function, often differing structurally by a single element or group, which may differ by modification of more than one group (e.g., 2, 3, or 4 groups) if it retains the same function or features (e.g., hydrophobicity) of said another chemical substance.

Said terpenoid TOC can be present in the water of the aquaculture system in a concentration ranging from about 10 to about 10000 ng/L. According to some embodiments, the concentration of said terpenoid ranges from about 10 to about 5000 ng/L. According to further embodiments, the concentration of said terpenoid ranges from about 10 to about 1000 ng/L. According to still further embodiments, the concentration of said terpenoid ranges from about 50 to about 750 ng/L or from about 100 to about 500 ng/L. In additional embodiments, the concentration of said terpenoid ranges from about 10 to about 100 ng/L, from about 100 to about 250 ng/L, from about 250 to about 500 ng/L, from about 500 to about 750 ng/L, from about 500 to about 100 ng/L, or from about 750 to about 1000 ng/L.

The terms “degrading”, “transforming” and “removing”, which are used herein interchangeably, refer, in some embodiments, to the conversion of the TOCs into compounds, which do not cause off-flavor and/or malodor in the aquaculture system. In further embodiments, the terms “degrading”, “transforming” and “removing” are meant to indicate that the concentration of the terpenoid noxious TOCs in the treated water is reduced to below 10 ng/L.

According to some currently preferred embodiments, the microorganisms, which are capable of degrading or transforming said terpenoid TOCs are endemic to the aquaculture system, which water is being treated. In certain embodiments, said microorganisms comprise gram negative bacteria. Non-limiting examples suitable microorganism species include Chryseobacterium sp., Sinorhizobium sp., Stenotrophomonas sp., Sphingopyxis alaskensis, sp., Novosphingobium stygiae sp., Pseudomonas veronii sp., Methylobacterium sp., Micrococcus sp., Flavobacterium sp., Brevibacterium sp., Pseudomonas sp., Pseudomonas sp., Rhodococcus wratislaviensis sp., Acinetobacter sp., as well as the species of the Oxalobacteraceae and Alphaproteobacteria families.

According to some embodiments, the carrier has a form of a particle. The term “particle”, as used herein, is understood to mean small discrete object, small discrete unit, or small discrete portion, and encompasses a bead, granule, grain, capsule and the like. Preferably, a particle is a solid or semi-solid unit, and can be spherical, spheroidal, oval, or polyhedral in shape, such as, for example, having cubic, hexahedron, tetrahedron, octahedron, dodecahedron, or icosahedron shape or have an irregular shape.

In some embodiments, the particle is essentially spherical. The term “essentially spherical”, as used herein, refers, in some embodiments, to a particle having an aspect ratio of from about 0.90 to about 1.

The aspect ratio can be calculated according to the following formula,

$$Aspect ratio = \frac{{D}_{max}}{{D}_{min}} \qquad Formula ( 1 )$$

wherein Dₘₐₓ is the largest length of the particle in the first dimension and Dₘᵢₙ is the smallest length of the particle in the second dimension. In some embodiments, the first dimension is perpendicular to the second dimension.

In further embodiments, the aspect ratio of the particle is at least about 0.90, at least about 0.91, at least about 0.92, at least about 0.93, at least about 0.94, or at least about 0.95.

The particle can have a particle size ranging from about 100 μm to about 10 cm. In some embodiments, the particle has a particle size ranging from about 1 mm to about 1 cm. The term “particle size”, as used in various herein, refers to the length of the particle in the longest dimension thereof. When the particle is spherical or near-spherical, the term “particle size” corresponds to the particle's diameter.

According to some embodiments, the hydrophobic agent is homogeneously mixed with the hydrocolloid.

According to some embodiments, the hydrocolloid is configured to entrap said hydrophobic agent. In some related embodiments, the hydrocolloid is a gelled hydrocolloid. As mentioned hereinabove, the hydrocolloid gel network, which supports and/or entraps the hydrophobic agent allows to maximize the surface area, which is available for the colonization of the terpenoid-degrading bacteria and the bulk, which is configured to adsorb the off-flavor terpenoid compounds.

According to further embodiments, the hydrophobic agent is disposed within the hydrocolloid. According to further embodiments, the hydrophobic agent is dispersed within the hydrocolloid. According to yet further embodiments, the hydrophobic agent is uniformly dispersed within the hydrocolloid. The hydrophobic agent can be in a form of small isles, micro-particles, nanoparticles, micro-bubbles, or nano-bubbles, dispersed throughout the hydrocolloid network.

According to some embodiments, the hydrocolloid is disposed within the hydrophobic agent. According to further embodiments, the hydrocolloid is dispersed within the hydrophobic agent. According to yet further embodiments, the hydrocolloid is uniformly dispersed within the hydrophobic agent. For example, the hydrocolloid can be in a form of micro- or nano-particles of a powder dispersed throughout the hydrophobic agent, wherein said hydrophobic agent comprises a wax or an oil-wax combination. Without wishing to being bound by theory or mechanism of action, it is contemplated that the presence of the hydrocolloid within the hydrophobic agent enhances the contact area between the hydrophobic agent and the water of the aquaculture system and enhances diffusion of said water into the bulk of the hydrophobic agent, thereby enhancing colonization efficiency of the terpenoid-degrading bacteria and the TOCs removal efficiency.

The terms “homogeneously” and “uniformly”, which are used interchangeably, denote that the volume percentage of the hydrophobic agent and/or the hydrocolloid varies from one position within the particle to another by less than 20%, preferably less than 10%. In still further embodiments, the particle is isotropic. The term “isotropic”, as used herein, refers to the independence of the concentration of the hydrophobic agent and/or hydrocolloid of a certain direction within the particle.

The hydrophobic agent can be thoroughly mixed with the hydrocolloid or can be homogenized to obtain smaller oil isles or bubbles or wax micro- or-nano-particles within the hydrocolloid network. Alternatively, the hydrophobic agent can be thoroughly mixed with the hydrocolloid or can be homogenized to obtain micro- or nano-particles of a hydrocolloid powder within the hydrophobic agent network. In additional embodiments, the hydrophobic agent comprises a first and a second hydrophobic agent (also termed herein “additional hydrocolloid”), wherein the first hydrophobic agent is mixed with the hydrocolloid and their mixture is then dispersed in the second hydrophobic agent. For example, the hydrocolloid can be mixed with oil and said mixture is then dispersed in a wax matrix.

According to some embodiments, the carrier has a form of a particle comprising a liquid core enclosed by a membrane. In further embodiments, said liquid core comprises the hydrophobic agent and the membrane comprises the hydrocolloid. In further embodiments, the liquid core consists of the hydrophobic agent and the membrane consists of the hydrocolloid, and, optionally, a cross-linking agent, as detailed herein below. In certain such embodiments, the hydrophobic agent forms the liquid core of the particle and the hydrocolloid forms the membrane thereof. The membrane can be solid or semi-solid.

According to some embodiments, the liquid core of the particle has a diameter ranging from about 10 μm to about 1 cm. According to some embodiments, the membrane of the particle has a thickness ranging from about 1 μm to about 5 mm. In further embodiments, the ratio between the diameter of the liquid core and the membrane thickness ranges from about 100,000:1 to 1:500.

The term “hydrophobic agent”, as used herein, refers to a molecule, substance, or composition, having a smaller electrostatic attraction towards water molecules than the electrostatic attraction between two water molecules. According to some embodiments, the hydrophobic agent has a water contact angle of above 90°. According to further embodiments, the hydrophobic agent has a water contact angle of above 100°. According to still further embodiments, the hydrophobic agent has a water contact angle of above 110°. According to yet further embodiments, the hydrophobic agent has a water contact angle of above 120°.

According to some currently preferred embodiments, the hydrophobic agent has a melting temperature below 70° C. According to further embodiments, the hydrophobic agent has a melting temperature below 65° C., below about 60° C., below about 50° C., or below about 45° C. Each possibility represents a separate embodiment of the invention. Suitable hydrophobic agents can be selected from oils, waxes, fatty acids, fatty alcohols, tocopherols, tocotrienols, and combinations and derivatives thereof.

In some exemplary embodiments, the hydrophobic agent comprises an oil. The oil can be selected from organic oils, mineral oils, silicone oils, and combinations thereof. While only edible and nontoxic oils can be used for removing noxious TOCs from aquaculture system adapted for growing aquaculture species for food purposes, in aquaculture systems containing species intended for other uses (e.g., decorative purposes), additional hydrophobic agents can be employed. Organic oils can include vegetable, plant and animal oils. Non-limiting examples of suitable vegetable and plant oils include soybean oil, coconut oil, cottonseed oil, peanut oil, rapeseed oil, canola oil, safflower oil, peanut oil, walnut oil, sesame oil, olive oil, linseed oil, evening primrose oil, sea buckthorn oil, palm oil, palm kernel oil, sunflower oil, corn oil, jojoba oil, marrow oil, grapeseed oil, hazelnut oil, apricot oil, macadamia oil, almond oil, castor oil, acai berry oil, apricot oil, avocado oil, baobab oil, black cumin oil, blackcurrant seed oil, blueberry seed oil, borage oil, camelina oil, cherry kernel oil, chia seed oil, cranberry seed oil, hemp seed oil, macadamia nut oil, marula oil, neem oil, oat oil, argan oil, pomegranate seed oil, peach kernel oil, plum kernel oil, barbary fig seed oil, raspberry seed oil, rice bran oil, rosehip seed oil, tamanu oil, wheatgerm oil, meadowfoam oil, flaxseed oil, perilla oil, oiticica oil, grapestone oil, shea butter, tung oil, wheat germ oil, and combinations thereof. Non-limiting examples of suitable animal oils include cod liver oil, fish oil, lard oil, mink oil, neatsfoot oil, tallow oil, woolgrease, and combinations thereof. The oil in the particle (or carrier) for use in the method of the present invention can be refined and/or hydrogenated.

In certain embodiments, said oil is a soybean oil. Soybean oil is a vegetable oil extracted from the seeds of the soybean (Glycine max). Advantageously, soybean oil is one of the most widely consumed cooking oils, being, therefore highly abundant and inexpensive. The major fatty acids in soybean oil include alpha-linolenic acid (C-18:3), linoleic acid (C-18:2), oleic acid (C-18:1), stearic acid (C-18:0), and palmitic acid (C-16:0). According to some embodiments, the soybean oil is a refined oil.

In certain embodiments, said oil is a sunflower oil. Sunflower oil is primarily composed of linoleic acid and oleic acid.

According to some embodiments, the hydrophobic agent comprises a wax. The wax can be selected from animal wax, insect wax, plant wax, mineral wax, silicone wax, and combinations thereof. Non-limiting examples of suitable animal or insect waxes include beeswax, lanolin, hard tallow, and combinations thereof. Non-limiting examples of suitable vegetable waxes include Japan wax, castor wax, sugar cane wax, candelilla wax, bayberry wax, cocoa butter, illipe butter, and combinations thereof. Non-limiting examples of suitable petroleum or mineral waxes include ceresin, petrolatum, paraffin, and combinations thereof.

According to some embodiments, the hydrophobic agent comprises a fatty alcohol. Non-limiting examples of suitable fatty alcohols include stearyl alcohol, cetyl alcohol, lanolin alcohol, 2-octyldodecanol, and combinations thereof.

According to some embodiments, the hydrophobic agent comprises a fatty acid. Non-limiting examples of suitable fatty acids include oleic acid, stearic acid, palmitic acid, lauric acid, myristic acid, behenic acid, isostearic acid, and combinations thereof.

According to some embodiments, the hydrophobic agent comprises Vitamin E.

The hydrophobic agent can include variations of the oils, waxes, fatty acids, fatty acid alcohols, fatty esters, tocopherols, and tocotrienols, as described hereinabove, including analogues, homologues and variations in molecular weight, molecular weight distribution, chain branching, copolymers and chain termination groups which can be readily optimized by those of ordinary skill in the art.

The hydrophobic agent can be present in the carrier or particle in a weight percent ranging from about 10% to about 99.5% out of the total wet weight of the carrier or particle. In some embodiments, the hydrophobic agent is present in the carrier or particle in a weight percent ranging from about 10% to about 99% out of the total wet weight of the carrier or particle. In further embodiments, the hydrophobic agent is present in the carrier or particle in a weight percent ranging from about 10% to about 95% out of the total wet weight of the carrier or particle. In still further embodiments, the hydrophobic agent is present in the carrier or particle in a weight percent ranging from about 10% to about 90% out of the total wet weight of the carrier or particle. In yet further embodiments, the hydrophobic agent is present in the carrier or particle in a weight percent ranging from about 10% to about 80% out of the total wet weight of the carrier or particle. In still further embodiments, the hydrophobic agent is present in the carrier or particle in a weight percent ranging from about 10% to about 70% out of the total wet weight of the carrier or particle. In yet further embodiments, the hydrophobic agent is present in the carrier or particle in a weight percent ranging from about 10% to about 60% out of the total wet weight of the carrier or particle. In still further embodiments, the hydrophobic agent is present in the carrier or particle in a weight percent ranging from about 10% to about 50% out of the total wet weight of the carrier or particle. In further embodiments, the hydrophobic agent is present in the carrier or particle in a weight percent ranging from about 10% to about 40% out of the total wet weight of the carrier or particle. In yet further embodiments, the hydrophobic agent is present in the carrier or particle in a weight percent ranging from about 15% to about 35% out of the total wet weight of the carrier or particle.

In certain embodiments, the hydrophobic agent is present in the particle in a weight percent of about 30% out of the total wet weight of the particle. In some related embodiments, the hydrophobic agent comprises soybean oil. It has been found by the inventors of the present invention that removal of geosmin and MIB increased with increasing the relative amount of oil in the particle up to a concentration of 40% (w/w). Surprisingly, while a steep increase in the TOC removal efficiency was observed with an increase in the oil concentration up to 30% (w/w), there was no significant improvement in the TOC adsorption efficiency, when increasing oil concentration from 30% (w/w) to 40% (w/w). Additionally, oil concentration higher than 30% (w/w) resulted in reduced compressive stress and compressive strain following 24 hours of particles' use.

The term “hydrocolloid”, as used herein refers to a hydrophilic polymer, which is at least partly water-soluble, said polymer being natural, synthetic, or semisynthetic, which preferably form gels or viscous solutions or suspensions in aqueous systems. Natural hydrocolloids typically belong to the protein or polysaccharide classes, with a large number of hydrocolloids originating from nature, in particular from land plants, algae, animals and bacteria. The hydrocolloids are nowadays widely used in a variety of industrial sectors to perform a number of functions including thickening and gelling aqueous solutions, stabilizing foams, emulsions and dispersion, inhibiting ice and sugar crystal formation and the controlled release of flavor. Hydrocolloids are often used as thickeners in cosmetics and products of the food industry. For further details on the term hydrocolloid, reference can in particular be made to G. O. Phillips and P. A. Williams. Handbook of Hydrocolloids. 2nd edition; CRC Press and Woodhead Publishing Limited, 2009; page 1, the content whereof relating to this is by reference completely included herein.

The term “hydrophilic polymer”, as used herein, refers to a polymer having a greater solubility in an aqueous medium than in a non-aqueous medium, e.g., by at least about 10%. In some embodiments, the hydrophilic polymer has a greater solubility in the aqueous medium than in the non-aqueous medium by at least about 20%, at least about 30%, at least about 40%, at least about 50%, at least about 60%, at least about 70%, at least about 80%, or at least about 90%. In some embodiments, the hydrophilic polymer has a greater solubility in the aqueous medium than in the non-aqueous medium by at least about 1.5-fold, including, e.g., at least about 2-fold, at least about 3-fold, at least about 4-fold, at least about 5-fold, at least about 6-fold, at least about 7-fold, at least about 8-fold, or at least about 9-fold. The temperature of the aqueous medium can range from about 15° C. to about 100° C.

According to some embodiments, the hydrocolloid can be selected from a natural hydrocolloid, a semisynthetic hydrocolloid, a synthetic hydrocolloid, and combinations thereof. According to further embodiments. the natural hydrocolloid is selected from a polysaccharide, oligosaccharide, protein, and protein-polysaccharide complex. The natural hydrocolloid can be obtained from various sources, such as, e.g., botanical, algal, microbial, or animal sources. According to some currently preferred embodiments, the hydrocolloid is a gelling hydrocolloid. Non-limiting examples of suitable natural hydrocolloids include alginate, agar, agarose, gelatin, low methoxy pectin (LMP), chitosan, gellan, carrageenan, locust bean gum (LBG), guar gum, carrageenan, arabinoxylan, cellulose, curdlan, furcellaran, gellan, β-glucan, starch, modified starch, gum arabic, gum tragacanth, tamarind gum, fenugreek gum, cassia gum, tara gum, xanthan, pullulan, egg protein, vegetable protein, dairy protein, and combinations thereof;

Non-limiting examples of semisynthetic hydrocolloids include methylcellulose and sodium carboxymethylcellulose. Some examples of synthetic hydrocolloids (also referred to as “polymers” including polymers, cross-linked polymers, and copolymers) include polyacrylates, such as, for example, polyacrylamide, high molecular weight polyethylene glycols and polypropylene glycols, polyethylene oxides, polyacrylic acid polymers (e.g., CARBOPOL), poly vinyl alcohols, and combinations thereof.

In some exemplary embodiments, the hydrocolloid comprises alginate. Alginate, also termed herein algin, is an anionic polysaccharide distributed widely in the cell walls of brown algae, where through binding with water it forms a viscous gum. Alginate has a linear polymeric structure composed of D-mannuronic acid (M block) and L-guluronic acid (G block). Alginate is both food and skin safe. The biggest advantage of alginates is its liquid-gel behavior in aqueous solutions. When monovalent ions (e.g., sodium in sodium alginate) are exchanged for divalent ions (especially calcium), the reaction proceeds almost immediately, changing from a low viscosity solution to a gel structure. The gelled mass is a copolymer composed of two kinds of monomer units.

Preferably, the hydrocolloid is a cross-linked hydrocolloid. The hydrocolloid can be cross-linked by a suitable cross-linking agent. in some embodiments, the hydrocolloid is cross-linked by an ion selected from, but not limited to, calcium, magnesium, potassium, barium, aluminum, copper, lead, strontium ion, and combinations thereof. The type of the cross-linking ion can be chosen based on the particular hydrocolloid, as well as the type of the species in the aquaculture system (e.g., edible or decorative). In some exemplary embodiments, the hydrocolloid is cross-linked by calcium ions. In further embodiments, the hydrocolloid comprises alginate, which is cross-linked by calcium ions.

Many hydrocolloids can be crosslinked or interact with poly-cations via polyanion-polycation reactions, which result in the creation of complexes that produce a gel Accordingly, in some embodiments, the hydrocolloid is cross-linked by a polycation. Non-limiting examples of suitable polycations include chitosan, poly(L-lysine), and polyethyleneimine (PEI).

The hydrocolloid can be present in the carrier or particle in a weight percent ranging from about 0.05% to about 10% out of the total wet weight of the carrier or particle. In some embodiments, the hydrocolloid is present in the carrier or particle in a weight percent ranging from about 0.1% to about 5% out of the total wet weight of the carrier or particle. In further embodiments, the hydrocolloid is present in the carrier or particle in a weight percent ranging from about 0.5% to about 5% out of the total wet weight of the carrier or particle.

In some exemplary embodiments, the hydrocolloid is present in the particle in a weight percent of about 1%-2% out of the total wet weight of the particle.

The cross-linking agent can be present in the carrier or particle in a weight percent ranging from about 0.1% to about 5% out of the total wet weight of the carrier or particle. In some embodiments, the cross-linking agent is present in the carrier or particle in a weight percent ranging from about 0.5% to about 2% out of the total wet weight of the carrier or particle. In further embodiments, the cross-linking agent is present in the carrier or particle in a weight percent ranging from about 1% to about 2% out of the total wet weight of the carrier or particle.

The term “total wet weight”, as used herein, refers to the weight of the carrier or particle, in which the hydrocolloid is in its gelled state. i.e., comprising large quantity of absorbed water. Typically, the hydrocolloid, which is in gelled state will be present in the outer portion of the carrier, for example, wherein the hydrophobic agent is dispersed within the hydrocolloid or wherein the carrier comprises a hydrophobic agent core and a hydrocolloid shell. In some embodiments, the carrier or particle comprises from about 50% (w/w) to about 99% (w/w) water out of the total wet weight of the carrier or particle. In further embodiments, the carrier or particle comprises from about 60% (w/w) to about 95% (w/w) of water out of the total wet weight of the carrier or particle. In yet further embodiments, the carrier or particle comprises from about 60% (w/w) to about 85% (w/w) of water out of the total wet weight of the carrier or particle. In still further embodiments, the carrier or particle comprises from about 65% (w/w) to about 75% (w/w) of water out of the total wet weight of the carrier or particle.

According to some embodiments, the carrier or particle are essentially devoid of water. The term “essentially devoid of water”, as used herein, refers in some embodiments, to a carrier or particle comprising less than about 0.1% (w/w) water out of the total dry weight of the carrier or particle. Typically, the carrier or particle, which is essentially devoid of water will contain a hydrocolloid, which is present in the inner portion of the carrier, for example, wherein the hydrocolloid is dispersed within the hydrophobic agent. In certain such embodiments, the hydrocolloid is not in a gelled state and does not contain large quantity of absorbed water (at least until the initial contact between the carrier and the aquaculture system water). Accordingly, the term “total dry weight”, as used herein, refers in some embodiments, to the weight of the carrier or particle, in which the hydrocolloid is not in its gelled state. In further embodiments, the term “total dry weight” refers to the weight of the carrier or particle, in which the hydrocolloid comprises less than about 0.1 g water per 1 g of the hydrocolloid. In further embodiments, the term “total dry weight” refers to the weight of the carrier or particle, which contain less than about 10% (w/w) water.

The hydrophobic agent can be present in the carrier or particle in a weight percent ranging from about 88% to about 99.5% out of the total dry weight of the carrier or particle. In some embodiments, the hydrophobic agent is present in the carrier or particle in a weight percent ranging from about 90% to about 99.5% out of the total dry weight of the carrier or particle. In further embodiments, the hydrophobic agent is present in the carrier or particle in a weight percent ranging from about 95% to about 99% out of the total dry weight of the carrier or particle.

The hydrocolloid can be present in the carrier or particle in a weight percent ranging from about 0.01% to about 2% out of the total dry weight of the carrier or particle. In some embodiments, the hydrocolloid is present in the carrier or particle in a weight percent ranging from about 0.1% to about 2% out of the total dry weight of the carrier or particle.

According to some embodiments, the particle (or the carrier) further comprises an emulsifier. The emulsifier can be selected from ethoxylated sorbitan esters of fatty acids (i.e., polysorbates), succinylated monoglycerides, sucrose esters of fatty acids, sucroglycerides, polyglycerol esters of fatty acids, polyglycerol polyricinoleate, propane-1,2-diol esters of fatty acids, propylene glycol esters of fatty acids, lactylated fatty acid esters of glycerol and propane-1, thermally oxidized soya bean oil interacted with mono- and diglycerides of fatty acids, dioctyl sodium sulfosuccinate, sodium stearoyl-2-lactylate, calcium stearoyl-2-lactylate, stearyl tartrate, stearyl citrate, sodium stearoyl fumarate, calcium stearoyl fumarate, sodium lauryl sulfate, ethoxylated mono- and di-glycerides, methyl glucoside-coconut oil ester, propane-1,2-diol, sorbitan monostearate, sorbitan tristearate, sorbitan monolaurate, sorbitan monooleate, sorbitan monopalmitate, sorbitan trioleate, polyoxypropylene-polyoxyethylene polymers, partial polyglycerol esters of polycondensed fatty acids of castor oil, and stigmasterol-rich plant sterols. In certain embodiments, the surfactant comprises a polysorbate. Non-limiting examples of suitable polysorbates include Polysorbate 80 (Tween 80), Polysorbate 20 (Tween 20), Polysorbate 40 (Tween 40), Polysorbate 60 (Tween 60), and Polysorbate 65 (Tween 65). In some exemplary embodiments, the surfactant is Tween 80.

The emulsifier can be present in the carrier or particle in a weight percent of up to about 1% of the total wet weight of the carrier or particle. In some embodiments, the emulsifier is present in the carrier or particle in a weight percent ranging from about 0.001% to about 1% out of the total wet weight of the carrier or particle. In further embodiments, the emulsifier is present in the carrier or particle in a weight percent ranging from about 0.01% to about 1% out of the total wet weight of the carrier or particle or from about 0.1% to about 1% out of the total wet weight of the carrier or particle.

The carrier or the particle can further include fillers, lubricants, pigments, plasticizers, and surface-tension reducing agents, as known in the art. Non-limiting examples of suitable fillers include bentonite and kaolin. The filler can be present in the carrier in a weight percent ranging from about 0.1% to about 10% out of the total wet or dry weight of the carrier or particle.

According to certain embodiments, the carrier or particle comprises from about 10% (w/w) to about 40% (w/w) hydrophobic agent; from about 0.1% (w/w) to about 5% (w/w) hydrocolloid; from about 0.1% (w/w) to about 5% (w/w) cross-linking agent; from about 0.001% (w/w) to about 1% (w/w) emulsifier; and from about 50% (w/w) to about 95% (w/w) water, out of the total wet weight of the carrier or particle. In some exemplary embodiments, the hydrophobic agent comprises an oil. In further exemplary embodiments, said oil is soybean oil. In additional exemplary embodiments, the hydrocolloid comprises alginate and the cross-linking agent comprises calcium ions.

According to certain embodiments, the carrier or particle comprises from about 88% (w/w) to about 99.5% (w/w) hydrophobic agent and from about 0.1% (w/w) to about 2% (w/w) hydrocolloid, out of the total dry weight of the carrier or particle. In some embodiments, the carrier or particle further comprises from about 0.1% (w/w) to about 10% (w/w) filler. In some embodiments, the hydrophobic agent comprises a combination of an oil and a wax. The wax can be present in the carrier or particle in a weight percent ranging from, about 50% (w/w) to about 89.5% (w/w). The oil can be present in the carrier or particle in a weight percent ranging from, about 10% (w/w) to about 40% (w/w). In some exemplary embodiments, said oil is sunflower oil and said wax is beeswax. In additional exemplary embodiments, the hydrocolloid comprises alginate. In further exemplary embodiments, the filler comprises bentonite.

The carriers (being in the form of particles) for use in the method of the present invention were found to be stable following contact with water containing geosmin and MIB. In particular, the concentration of the hydrophobic agent within the particle was found to stay essentially unchanged and the particle was mechanically stable following contact with water containing geosmin and MIB. Preferably, the particle maintains at least about 65% of its compressive strain and about 25% of its compressive stress while being in contact with the water of the aquaculture system for about 6 weeks.

According to some embodiments, the particle is a core-shell particle, comprising a solid or semi-solid core comprising the hydrophobic agent (also termed herein “first hydrophobic agent”), the hydrocolloid (also termed herein “first hydrocolloid”), and, optionally, an emulsifier, and a water-permeable shell. The water-permeable shell can comprise a hydrocolloid (also termed herein “second hydrocolloid”). The first hydrocolloid and the second hydrocolloid can be the same or different. The water-permeable shell can further comprise a hydrophobic agent (also termed herein “second hydrophobic agent”). The first hydrophobic agent and the second hydrophobic agent can be the same or different.

According to some embodiments, the microorganisms which are capable of degrading said noxious TOCs are not present in the bulk of the particle. According to further embodiments, the particle does not contain the microorganisms which are capable of degrading said noxious TOCs before the contact with water is initiated. As explained hereinabove, the unique composition and structure of the particle (or carrier) affords for the formation of a population of microorganisms, which are endemic to the aquaculture system and which are capable of degrading the TOCs, on the particle surface, such that the particles can be manufactured without growing the microorganisms therewithin. When trying to grow and maintain the microorganisms within the bioactive carrier, the inventors have unexpectedly found that only small population with limited biomass can be effectively grown. Additionally, it was difficult to control the structure of the population, (i.e. which species are present and at which relative abundance) for the desired period of time. Accordingly, the present invention provides an elegant and cost- and time-efficient solution to an obstacle encountered during the development of carriers containing biological species for the removal of noxious TOCs from aquaculture systems.

According to other embodiments, the microorganisms which are capable of degrading the noxious TOCs are present within the carrier before the contact with water is initiated.

The particle (or carrier) suitable for use in the method of the present invention can be manufactured by mixing, e.g., by homogenization, of a hydrophobic with a hydrocolloid solution. For example, the hydrophobic agent being in a liquid form, can be added to an aqueous solution comprising the dissolved hydrocolloid. Alternatively, the aqueous solution comprising the hydrocolloid can be added to the hydrophobic agent being in a liquid form. The aqueous solution can include from about 0.5 to about 5% (w/w) of the hydrocolloid. The mixture can be agitated to facilitate formation of a uniform mixture. Additionally or alternatively, an emulsifier can be added to the mixture of the hydrophobic agent and the hydrocolloid to induce formation of a homogeneous emulsion.

According to some embodiments, said mixture comprises from about 10 to about 60% (w/w) of the hydrophobic agent. According to some embodiments, said mixture comprises from about 0.01 to about 10% (w/w) of the hydrocolloid. According to some embodiments, said mixture comprises from about 0.1 to about 10% (w/w) of the emulsifier. According to some embodiments, said mixture comprises from about 0.1 to about 10% (w/w) of the emulsifier. According to further embodiments, said mixture comprises from about 0.5 to about 5% (w/w) of the emulsifier. According to yet further embodiments, said mixture comprises about 1% (w/w) of the emulsifier.

The mixture of the hydrophobic agent, the hydrocolloid, and, optionally, the emulsifier, can be added to an aqueous solution comprising a cross-linking ion. The cross-linking ion can be present in said aqueous composition in a concentration ranging from about 0.1 to 20% (w/w). In some embodiments, the mixture is dropped (or dripped) into the cross-linking solution. In some embodiments, the volume of the dripped mixture of the hydrophobic agent, the hydrocolloid, and, optionally, the emulsifier ranges from about 1 μL to about 20 mL. According to some embodiments, the manufacturing process further comprises separating the obtained particles from the cross-linking solution. It is to be understood that the carrier or the particle can be dried before the step of contacting it with water from the aquaculture system, for example, for storage purposes. The drying can be performed by any suitable techniques as known in the art, for example by sun drying, drying at ambient temperatures, vacuum drying, fluidized bed drying, freeze dehydration (lyophilization), and any combination thereof. According to some embodiments, the manufacturing process comprises forming a water-permeable shell comprising a second hydrocolloid, for example, by suspending the obtained carrier in a solution comprising the second hydrocolloid. Said second hydrocolloid can be crosslinked with a suitable-crosslinking agent. The solution comprising the second hydrocolloid can further include a second hydrophobic agent mixed with the hydrocolloid, and, optionally, a second emulsifier.

According to some embodiments, the manufacturing process comprises mixing the hydrophobic agent with the hydrocolloid in its dry form. In some embodiments, the hydrophobic agent comprises a combination of a wax and an oil. In further embodiments, the wax is melted and then mixed with the oil. According to further embodiments, the hydrocolloid is dried before the mixing. According to some embodiments, the process further includes adding a filler to the wax-oil mixture, which is then followed by the addition of the hydrocolloid. The manufacturing process can further include dropping the mixture (preferably, wherein the mixture is heated to about 50-90° C.) into cold or room temperature water to form the carriers.

In some embodiments, said manufacturing process does not include a step of growing microorganisms, such as bacteria, within the bulk of the carrier or particle or on the particle surface.

In other embodiments, the manufacturing process includes a step of growing microorganisms, such as bacteria, within the bulk of the carrier or particle or on its surface. In additional embodiments, the manufacturing process includes a step of introducing microorganisms, such as bacteria, into the bulk of the carrier or particle or upon its surface.

The hydrophobic agent, hydrocolloid, emulsifier and cross-linking agent can be selected as detailed in the embodiments relating to the carrier composition hereinabove.

According to some embodiments, the hydrophobic agent, which is mixed with the hydrocolloid is a soybean oil.

According to some embodiments, the hydrocolloid in the aqueous solution is alginate. According to some embodiments, alginate is selected from alginic acid, an ester of alginic acid, an alginate salt and combinations thereof. The ester of alginic acid can include polypropylene glycol alginate (PGA). The alginate salt can be selected from sodium, potassium, and ammonium salts, and combinations thereof. In some exemplary embodiments, the alginate is sodium alginate. According to some embodiments, the emulsifier, which is mixed with the hydrophobic agent and the hydrocolloid is Tween 80.

The carrier can be prepared as a large batch, e.g., a large rectangular slab, which is further divided into smaller particles or pieces, having any desired shape.

The method of the present invention for the removal of noxious TOCs can be used in any aquaculture system, as known in the art, and in particular these with high levels of geosmin and MIB. Aquaculture systems in which the method of the invention can be beneficially employed include, inter alia, a recirculating aquaculture system (RAS), a raceway fish farm, multi-trophic aquaculture system, aquaponics system, an aquaculture species pond, an aquaculture species pool, an aquaculture species container, an aquaculture species tank, a live aquaculture species transportation apparatus, and an aquaculture depuration system. The aquaculture system can be a marine aquaculture system or a fresh water aquaculture system.

According to some embodiments, the aquaculture is a recirculating aquaculture system. The term “recirculating” refers to the fact that as low as 1% of water is exchanged per day, or even less. RAS system types vary but can include clear-water RAS which have external biofilters that contain a microbial community on plastic biomedia, and biofloc systems which have a dense microbial community suspended in the water column. According to some embodiments, the RAS includes at least one aquaculture species reservoir, such as, but not limited to, a tank, basin, or pond. The volume of said reservoir can range from about 0.1 to about 500 m³. According to some embodiments, the volume of said reservoir ranges from about 1 to about 100 m³. The RAS can further include at least one filter selected from a mechanical filter, solids removal filter, biological filter, and nitrification unit.

The method of the present invention is applicable to any aquaculture species, including aquatic vertebrate and invertebrate. Non-limiting examples of the aquaculture species, which can be present in the aquaculture system include fish, shrimp, prawns, mussels, oysters, crab, lobster, scallop, conch and eel.

According to some embodiments, the method for the removal of noxious TOCs comprises contacting the water of the aquaculture system with a plurality of said carriers. In certain embodiments, the plurality of carriers comprises a plurality of particles. The term “plurality”, as used herein, is meant to encompass two or more carriers or particles. The plurality of carriers can be contained or immobilized within a receptacle. The term “immobilized” refers to carriers that have been arranged in such a manner so they will not move significantly when the aquaculture water comes into contact with the carriers. Said immobilization of the plurality of carriers enables flow of the aquaculture water through the receptacle. The receptacle can include a mesh bag, a stacked packed column, a reactor, or any other holding device. The receptacle can be disposed within the aquaculture system.

The receptacle can comprise an inlet and an outlet, suitable for passing the water therethrough. In some embodiments, the sieve has pores which are smaller than the size of the carriers. In further embodiments, the sieve has pores smaller than about 2 mm. In still further embodiments, the sieve has pores smaller than about 0.5 mm. Immobilization of the carriers can be done within a receptacle having a sieve dividing the container into two parts, a first part comprising the carriers and a second part devoid of the carriers. The water to be treated flows through the first part, coming into contact with the carriers, and then flows to the second part, while the carriers due to their size are retained in the first part of the receptable by the sieve. The receptacle may have an inlet in the first part for entrance of the water to be treated and an outlet in the second part for the exit of the treated water.

According to some embodiments, the receptacle comprises a reactor. Non-limiting examples of the reactors, which can be used in accordance with the method of the present invention include a packed bed reactor (also termed herein “column” and “packed column”), fixed bed reactor, moving bed reactor, rotating bed reactor, and fluidized bed reactor.

According to some embodiments, the reactor comprises a column. The carriers can be closely packed within the column to allow aquaculture water passing there through, when in use. Flow modifiers can be introduced among the carriers to facilitate free flow of aquaculture water within the column.

According to some embodiments, the contacting step comprises extracting a portion of water (also termed herein “water to be treated”) from the aquaculture system and contacting said portion with the plurality of carriers. The water to be treated can be extracted from the aquaculture system by any suitable combination of tubes, valves and pumps. Said portion of water can be contained within the aquaculture system (but separately from the aquaculture species reservoir) to be treated internally. In some currently preferred embodiments, the at least one reservoir is in fluid flow connection with the receptacle. Alternatively, said portion of water is transferred from the aquaculture system, to be treated externally.

According to further embodiments, contacting the water to be treated with the plurality of carriers comprises flowing, passing or circulating the water to be treated through the plurality of carriers. The water can be flown through the plurality of carriers in a vertical or horizontal direction, or at any angle between said vertical and horizontal directions. The water can further be flown through the plurality of carriers in a linear fashion, including upflow and downflow directions, as well as in a circular fashion. As mentioned hereinabove, the carriers can be contained or immobilized in a receptacle. The water can be circulated through said receptacle by any suitable pump, as known in the art. The flow rate of the water to be treated can be adjusted based on the desired contact time between the water and the plurality of carriers.

The contact between the water and the plurality of carriers is maintained for a time period sufficient to allow the carriers to adsorb and degrade the noxious TOCs from the aquaculture system water. As would be apparent to those skilled in the art, the length of said time period depends on the volume of the water, amount of the carriers, the type of the receptacle, flow direction and fashion and/or flow rate of the water to be treated through the receptacle and can be readily evaluated in accordance with particular set-up conditions or as detailed in some exemplary embodiments hereinbelow.

In some exemplary embodiments, the contacting step comprises flowing the portion of water of the aquaculture system through a column comprising the plurality of carriers. The column can be a tubular vessel, wherein the portion of water is flown through the column in an upflow fashion. In some embodiments, volume of the column is at least about 0.5 L. The volume of the column can range from about 0.5 L to about 500,000 L. In some embodiments, the volume of the column ranges from about 1 L to about 100,00 L. In further embodiments, the volume of the column ranges from about 1 L to about 50,000 L. In further embodiments, the volume of the column ranges from about 1 L to about 10,000 L. In some embodiments, the volume of the column ranges from about 1 L to about 100 L. In some exemplary embodiments, the volume of the column is about 1 L.

According to some embodiments, the column comprises from about 1 g to about 100,000 kg of the carriers. According to further embodiments, the column comprises from about 1 g to about 50,000 kg of the carriers. According to some embodiments, the column comprises at least about 0.1 kg of the carriers. According to further embodiments, the column comprises from about 0.1 kg to about 10,000 kg of the carriers. According to still further embodiments, the column comprises from about 0.1 kg to about 2,500 kg of the carriers. According to additional embodiments, the column comprises from about 1 g to about 5 kg of the carriers. In further embodiments, the column comprises from about 10 g to about 100 g of the carriers. In some exemplary embodiments, the column comprises about 50 g carriers per 1 L volume of the column.

When a column is used, the flow rate of water through the column determines the “hydraulic retention time (HRT)” of the water in the column bed, and thus the contact time. According to some embodiments, the hydraulic retention time in said column ranges from about 0.1 to about 72 hours. According to further embodiments, the hydraulic retention time in said column ranges from about 0.1 to about 48 hours. According to till further embodiments, the hydraulic retention time in said column ranges from about 0.1 to about 20 hours. According to some embodiments, the hydraulic retention time ranges from about 3 to about 8 hours. According to certain embodiments, the hydraulic retention time is about 5 hours.

In additional exemplary embodiments, the contacting step comprises flowing the portion of water of the aquaculture system through a reactor comprising the plurality of carriers. In further embodiments, the portion of water is flown through the reactor in a circular fashion. In certain embodiments, the reactor is a moving bed reactor. In some related embodiments, the reactor is spherical and the water is circulated within the reactor by means of an immersed pump.

According to some embodiments, the carriers occupy up to about 75% of the reactor volume.

The column and/or the reactor can be designed to allow sufficient contact time (or hydraulic retention time) for the efficient removal of the noxious TOCs. Additionally or alternatively, the portion of water can be reintroduced to the column or the reactor several times, in order to further reduce the TOCs concentration.

Preferably, the treated water (i.e., the portion of water, which has been contacted with or flown through the plurality of carriers), is reintroduced to the aquaculture system. Concentration of the TOCs in the water can be monitored continuously or periodically and once it reaches the desired level, the treated water can be flown back to the aquaculture species reservoir by a suitable combination of valves, pumps, tubes and a control system. According to some currently preferred embodiments, the aquaculture system is a RAS and the portion of water to be treated is continuously circulated between the aquaculture species reservoir and the reactor comprising the plurality of carriers.

According to some currently preferred embodiments, the method of the invention and/or the contacting step comprises a preliminary sub-step, comprising contacting the plurality of carriers with a sample portion of the water from the aquaculture system, wherein said preliminary sub-step enables colonization of the microorganisms on the carrier surface prior to the contacting step, which is configured to adsorb and degrade the noxious TOCs. The preliminary sub-step can be a part of the contacting step, wherein contacting the plurality of carriers with the aquaculture system water first results in the colonization of the suitable microorganisms on the carriers' surface and then in adsorption of the TOCs on the carriers' surface and their degradation by the surface-bound microorganisms. The contacting step is typically performed immediately after the preliminary sub-step. The preliminary sub-step can be performed by flowing the water of the aquaculture system through the plurality of carriers contained in the receptacle by methods described hereinabove.

According to some embodiments, the preliminary sub-step comprises incubating the plurality of carriers with the sample portion of water, which contains sufficient amount of the TOCs-degrading bacteria. In certain embodiments, the ratio between the number of carriers and the volume of the aquaculture system water ranges from about 50 carriers/L to about 1000 carriers/L. In further embodiments, said ratio ranges from about 100 carriers/L to about 500 carriers/L. In some exemplary embodiments, said ratio is 50 carriers/L.

The preliminary sub-step can be performed for a time period sufficient for the formation of the microorganism population on the carrier surface. According to some embodiments, the preliminary sub-step is performed for at least about 24 hours. According to further embodiments, the preliminary sub-step is performed for at least about 48 hours, at least about 4 days, at least about 1 week, at least about 2 weeks, or at least about 1 month. Each possibility represents a separate embodiment of the invention. In certain embodiments, the preliminary sub-step is performed for at least about 2 weeks. Preferably, the preliminary sub-step is performed until the microorganism population growth reaches stationary phase.

The method for the removal of noxious TOCs can be effected under aerobic, as well as anaerobic conditions. In some embodiments, the contacting step is effected under aerobic conditions. In other embodiments, the contacting step if effected under anaerobic conditions.

One of additional advantages of the present method is that is also facilitates denitrification of the aquaculture system water. Source of nitrogen in aquaculture system is from feed proteins. Ammonium and ammonia exist in equilibrium in water; higher pH, higher temperature, and lower salinity all favor the ammonia compound which is highly toxic. To remove ammonia in RAS, the microbial process of nitrification is usually employed, which results in accumulation of nitrate, which is the end product of nitrification. Nitrate is much less toxic than either ammonia or nitrite; however, high concentrations reduce aquaculture species growth and can decrease survival. According to some embodiments, the method for the removal of noxious TOCs from the aquaculture system water results in the reduction of nitrate to elemental nitrogen via denitrification. The contact time between the plurality of carriers and the water to be treated can be adjusted based on the desired final levels of nitrate in the aquaculture system. In some embodiments, the method comprises a step of adjusting the nitrate concentration of the treated water, for example, to its initial concentration (prior to the treatment). The pH of the treated water can also be adjusted to the acceptable level.

As mentioned hereinabove, the invention further provides a recirculating aquaculture system (RAS) for maintaining aquaculture species, comprising, inter alia, a plurality of carriers comprising a hydrophobic agent configured to adsorb noxious taste or order compounds (TOCs), and a hydrocolloid wherein the carriers are adapted for the colonization of microorganisms which are capable of degrading or transforming said TOCs.

The composition of the carriers suitable for use in the recirculating aquaculture system of the present invention, concentration of the ingredients, other properties of the carriers, e.g., particle size, and their manufacturing process can be the same as detailed hereinabove in connection with the embodiments relating to the method of TOCs removal.

According to some embodiments, the RAS further comprises a reactor being in fluid flow connection with the reservoir and means for directing flow of a portion of the water from the reservoir to the reactor. In certain such embodiments, the plurality of carriers are disposed within the reactor.

The number of carriers within the reactor should be proportional to the reactor's volume. In some embodiments, the reactor contains at least 100 carriers per 1 L of the reactor's volume. In further embodiments, the reactor contains at least 200 carriers per 1 L of the reactor's volume. In still further embodiments, the reactor contains at least 500 carriers per 1 L of the reactor's volume. In additional embodiments, up to about 75% of the reactor's volume is filled with the carriers according to the various embodiments detailed herein.

The plurality of carriers can be immobilized within the reactor. Said immobilization of the plurality of carriers enables flow of the RAS water through the reactor, wherein the carriers do not move significantly when the water flows therethrough.

Preferably, the reactor comprises an inlet and an outlet, suitable for passing the water therethrough, wherein the water flows through the reactor in an upflow, downflow or horizontal fashion or at any suitable angle, as known in the art.

Non-limiting examples of suitable reactors, include packed bed reactors, fixed bed reactors, trickle-bed reactors, moving bed reactors, rotating bed reactors, and fluidized bed reactors.

A packed bed reactor can include a hollow tube, pipe, or other vessel that is filled with the plurality of carriers. The purpose of a packed bed is typically to improve contact between two phases in a chemical or similar process. The packed bed reactor (or column) can be filled with random dumped packing material (i.e., the plurality of carriers, thereby creating a random packed column). The packed bed reactor can further contain flow modifiers, such as, for example, small objects like Raschig rings. The packed bed reactor can further have specifically designed structured packing including structured packing sections, which are arranged or stacked (creating a stacked packed column). The volume of the column can range from about 1 to about 50,000 L.

A fixed bed reactor can include a cylindrical tube filled with the plurality of carriers, wherein the water flows through the particle bed and being converted into products. The particles can be present in multiple configurations including one large bed, several horizontal beds, several parallel packed tubes or multiple beds in their own shells. The water flow through the fixed bed reactor is typically downward.

A moving bed reactor generally includes a reactor, containing inherent carriers covered by a bacterial biofilm, in which the fluid to be treated passes up through or can be circulated within the reactor. In certain embodiments, the moving bed reactor is spherical and the water is circulated within the reactor by means of an immersed pump. The volume of the moving bed can range from about 10 to about 50,000 L.

A rotating bed reactor (RBR) typically holds a packed bed fixed within a receptacle with a central hole. When the receptacle is spinning immersed in a fluid phase, the inertia forces created by the spinning motion forces the fluid outwards, thereby creating a circulating flow through the rotating packed bed.

A fluidized bed reactor can include small particles, which are suspended by the upward motion of the water to be treated.

The RAS further includes the following components: a reservoir holding water and aquaculture species; at least one of solids removing filter, mechanical filter, and biological filter; a nitrification unit; and means for directing flow of a portion of the water from the reservoir to the reactor.

The reservoir (also termed herein “aquaculture species reservoir”) can be selected from, but not limited to, a tank, basin, or pond. The volume of said reservoir can range from about 0.1 to about 500 m³. According to some embodiments, the volume of said reservoir ranges from about 0.5 to about 250 m³. According to further embodiments, the volume of said reservoir ranges from about 1 to about 100 m³.

The reservoir can hold any aquaculture species, including aquatic vertebrate and invertebrate. Non-limiting examples of the aquaculture species, which can be present in the reservoir of the aquaculture system include fish, shrimp, prawns, mussels, oysters, crab, lobster, scallop, conch and eel.

According to some embodiments, the RAS includes at least one of the solids removal filter and the mechanical filter. According to some embodiments, the RAS includes at least one biological filter. According to some currently preferred embodiments, the RAS includes at least one of the solids removal filter and the mechanical filter and at least one biological filter.

The biological filter enables conversion of ammonia and ammonium excreted by the aquaculture system species into nitrate via nitrite. Accordingly, the biological filter can be configured to provide a substrate for the bacterial community, which can result in a biofilm growing within the filter. The biofilter can include any suitable nitrifying bacteria, as known in the art, that convert ammonia into nitrate via nitrite. According to some embodiments, the RAS further comprises means for directing flow of a portion of the water from the reservoir to the at least one biological filter.

The solids removal filter and/or the mechanical filter are configured to remove the solid waste from the system. Removing solids reduces bacteria growth, oxygen demand, and proliferation of diseases. Typical RAS solids removal filter comprises a sand filter or particle filter where solids become lodged and can be periodically back-flushed out of the filter. Another suitable filter type comprises a mechanical drum filter where water is run over a rotating drum screen that is periodically cleaned by pressurized spray nozzles, and the resulting slurry is treated or sent down the drain. In order to remove extremely fine particles or colloidal solids, a protein fractionator may be used with or without the addition of ozone. According to some embodiments, the RAS further comprises means for directing flow of a portion of the water from the reservoir to the at least one solids removal filter and/or mechanical filter.

According to certain embodiments, the reservoir, at least one of the solids removal filter and mechanical filter, the biological filter, and the reactor with the plurality of carriers are fluidly connected, such that a portion of the water can flow from the reservoir to the solids removal filter and/or the mechanical filter; from the solids removal filter and/or mechanical filter to the biological filter; from the biological filter to the reactor with the plurality of carriers; and from the reactor back to the reservoir. The reactor can be alternatively positioned between the solids removal filter and/or the mechanical filter and the biological filter, such that a portion of the water can flow from the reservoir to the solids removal filter and/or the mechanical filter; from the solids removal filter and/or mechanical filter to the reactor with the plurality of carriers; from the reactor with the plurality of carriers to the biological filter; and from the biological filter back to the reservoir.

The RAS according to the principles of the present invention can further include at least one of nitrification unit, oxygenation unit, pH control unit, temperature control unit, and water treatment unit. The nitrification unit may include any nitrifying entity capable of removing ammonia, either by biological, chemical, or physical means. For example, it may include nitrifying bacteria or an appropriate mineral used to adsorb ammonia, such as a zeolite. The oxygenation unit can perform reoxygenation of the RAS water via aeration and oxygenation. In aeration air can be pumped through an air stone or similar device that creates small bubbles in the water column, this resulting in a high surface area where oxygen can dissolve into the water. Additionally or alternatively, the water can be oxygenated by pumping in pure oxygen. pH control unit is configured to maintain the pH of the RAS water in a suitable range. pH control unit is typically operated to introduce alkaline compounds to the portion of the circulating water, such as, but not limited to, lime (CaCO₃) or sodium hydroxide (NaOH). pH can also be controlled by degassing CO₂ in a packed column or with an aerator. Temperature control unit can include at least one of a submerged heater, heat pump, chiller, and heat exchanger. The water treatment unit is configured to reduce the number of free-floating virus and bacteria in the RAS water. The water treatment unit can include an Ultra Violet (UV) and/or ozone water treatment system.

According to some embodiments, the RAS further comprises means for directing flow of a portion of the water from the reservoir to said additional units. The units can further be in fluid flow connection with the solids removal filter, mechanical filter, biological filter and/or the reactor with the plurality of carriers, and can be arranged along the recirculating water line as known in the art.

The means for directing a portion of water from the reservoir and between the different units of the RAS system, including, the reactor with the plurality of carriers, solids removal filter, mechanical filter, nitrification unit, oxygenation unit, pH control unit, temperature control unit, and/or water treatment unit can be selected from pumps, tubes, valves, control units, and any combination thereof.

As mentioned hereinabove, the present invention further provides is a bioactive filter comprising a plurality of carriers comprising a hydrophobic agent configured to adsorb noxious taste or order compounds (TOCs), and a hydrocolloid, wherein the carriers are adapted for the colonization of microorganisms which are capable of degrading or transforming said TOCs, wherein said bioactive filter is for use in the removal of noxious taste or odor compounds (TOCs) from water of an aquaculture system.

The composition of the carriers suitable for use in bioactive filter of the present invention, concentration of the ingredients, other properties of the carriers, e.g., particle size, and their manufacturing process can be the same as detailed hereinabove in connection with the embodiments relating to the method of TOCs removal.

In some embodiments, the bioactive filter comprises a body, an inlet and an outlet, wherein the inlet and outlet are suitable for passing the water therethrough, wherein the water flows through the bioactive filter body in an upflow, downflow or horizontal fashion or at any suitable angle, as known in the art. The bioactive filter can be in a form of a reactor, selected from, but not limited to, a packed bed reactor, fixed bed reactor, trickle-bed reactor, moving bed reactor, rotating bed reactor, and fluidized bed reactor, as detailed hereinabove. According to certain embodiments, the bioactive filter is in a form of a packed bed reactor (i.e., a column).

The number of carriers within the bioactive filter should be proportional to the filter's volume. In some embodiments, the bioactive filter contains at least 100 carriers per 1 L of the filter's volume. In further embodiments, the bioactive filter contains at least 200 carriers per 1 L of the filter's volume. In still further embodiments, the bioactive filter contains at least 500 carriers per 1 L of the filter's volume. The plurality of carriers can be immobilized within the bioactive filter. Said immobilization of the plurality of carriers enables flow of the water to be treated through the filter, wherein the carriers do not move significantly when the water flows therethrough.

As used herein and in the appended claims the singular forms “a”, “an,” and “the” include plural references unless the content clearly dictates otherwise. Thus, for example, reference to “particle” includes a plurality of such particles and equivalents thereof known to those skilled in the art, and so forth. It should be noted that the term “and” or the term “or” is generally employed in its sense including “and/or” unless the content clearly dictates otherwise.

As used herein, the term “about”, when referring to a measurable value such as an amount, a temporal duration, and the like, is meant to encompass variations of +/−20%, more preferably +/−5%, even more preferably +/−1%, and still more preferably +/−0.1% from the specified value, as such variations are appropriate to perform the disclosed methods.

The following examples are presented in order to more fully illustrate some embodiments of the invention. They should, in no way be construed, however, as limiting the broad scope of the invention. One skilled in the art can readily devise many variations and modifications of the principles disclosed herein without departing from the scope of the invention.

### EXAMPLES

### Example 1: Preparation of Oil-Hydrocolloid Carriers

Carriers comprising up to 40% (w/w) oil were prepared by emulsification of purified edible soybean oil in a hydrocolloid solution containing 1% (w/w) of emulsifier (Tween 80, Sigma). The hydrocolloid was alginate with a molecular mass of 60-70 kDa, containing 61% mannuronic acid and 39% guluronic acid (Sigma, LV, St. Louis, MO), which was dissolved in distilled water (1% w/w). The emulsified solution was dropped into a stirred cross-linking solution containing 1% (w/w) calcium chloride until the carriers were formed (about 30 minutes). This procedure resulted in a spontaneous cross-linking reaction that produced spherical carriers (about 15 carriers per 1 ml dripping solution) with an average diameter of about 4 mm.

### Example 2: Effect of Oil Contents of Carriers on Geosmin and MIB Removal

Removal of geosmin and MIB by the carriers synthesized in Example 1 was tested using carriers with different oil content. Freshly prepared carriers with oil content of 0, 10, 20, 30, and 40% (w/w) were aseptically incubated (to avoid or at least minimize biological activity) with sterile 50 ml of DDW (double distilled water) supplemented with either geosmin or MIB in serum bottles, sealed by air-tight butyl septa (to prevent TOCs vaporization). Using a magnetic stirrer all samples were gently mixed for 24 hours at room temperature.

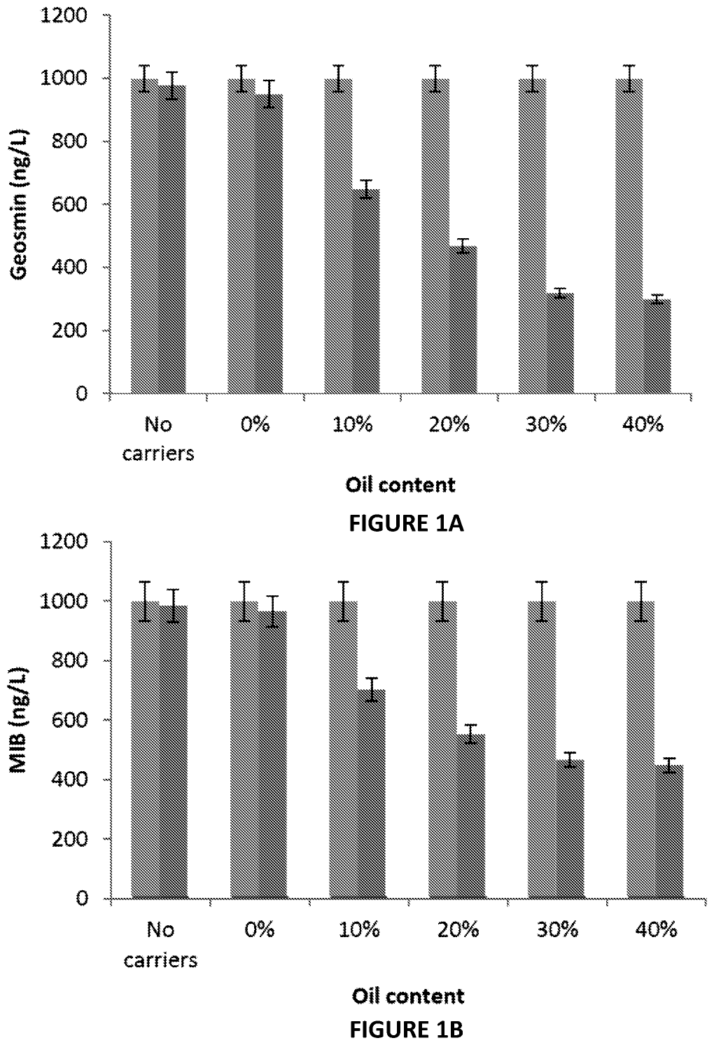

After 24 hours of incubation, TOCs concentration in the water was measured by GC-FID (Gas Chromatography with Flame Ionization Detector). It was found that removal of both compounds from deionized water increased with increasing levels of oil in the carriers up to a concentration of 30% (FIGS. 1A and 1B). No significant increase in the TOCs' adsorption efficiency of the carriers containing 40% (w/w) oil as compared to these with 30% (w/w) was observed. Additionally, a significant decline in the mechanical strength of the 40% oil-containing carriers was detected following 24 hours of incubation, as compared to the 30% oil-containing carriers.

### Example 3: Effect of Water from an Aquaculture System on Geosmin and MIB Removal

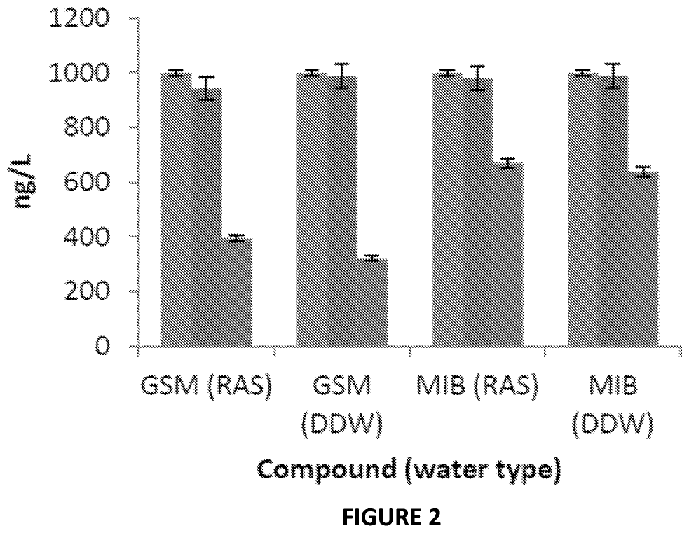

Carriers with 30% (w/w) oil content were aseptically incubated with either 50 ml of sterile double distilled water or water obtained from a recirculating aquaculture system. They were added to the incubation vessel at a concentration 200 carriers/L. Geosmin and MIB levels were determined at the beginning of the trial (prior to carriers' addition) and after 24 hours of incubation. In order to avoid any biological activity, water from the aquaculture system was treated by ultra-filtration and sterilization. It was found (FIG. 2) that geosmin and MIB adsorption by the carriers in the water derived from an aquaculture system was similar to that in distilled water.

### Example 4: Effect of Adsorbed Microorganisms on Geosmin and MIB Removal by Oil-Hydrocolloid Carriers

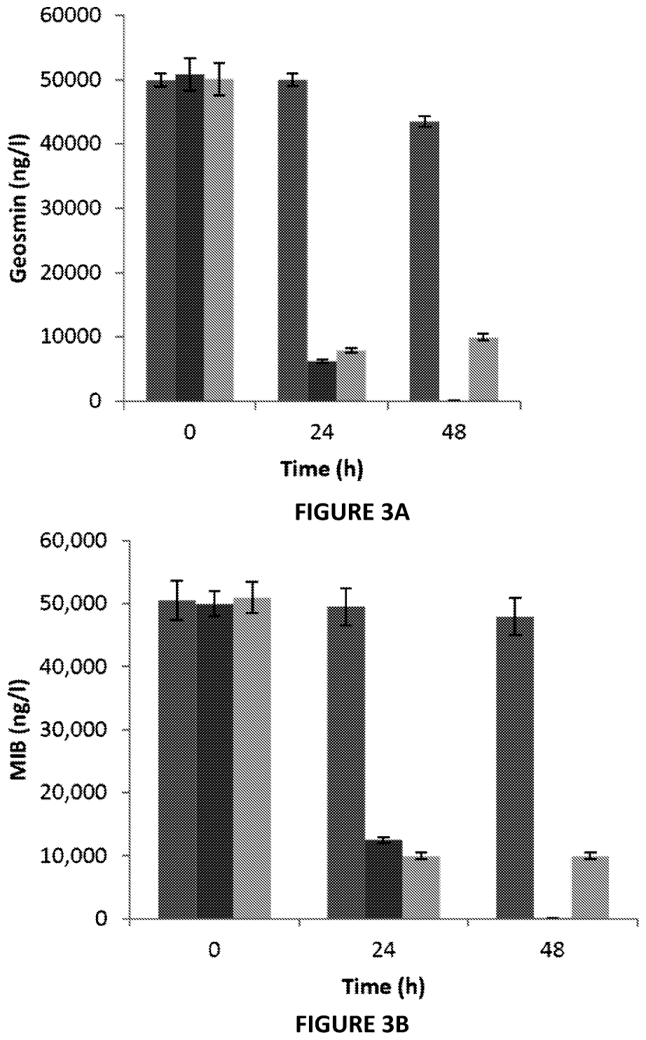

Microbial degradation of geosmin and MIB was examined by preincubation of the carriers with 30% (w/w) oil content in double distilled water containing either geosmin or MIB at relatively high concentrations (50,000 ng/L). Following 24 h of incubation under these conditions, the carriers were incubated in medium containing crude sludge from a recirculating aquaculture system, known to contain geosmin and MIB degrading bacteria. After 24 h, the carriers were washed and incubated in air-tight serum bottles, using a magnetic stirrer for gentle mixing in the presence of geosmin and MIB for 48 h. Standard carriers with 30% oil, which were not exposed to sludge were used as controls. An additional control, without carriers, was used to account for other removal processes (adsorption to septa, volatilization). It was found that, as opposed to carriers not exposed to sludge, complete removal of both geosmin and MIB from water was found when using carriers exposed to sludge after 48 h of incubation (FIGS. 3A and 3B).

The microbial composition of the carriers was determined by means of metagenomic analyses. Evidence was found for the development of bacteria on the surface of the carriers, capable of both nitrate reduction and geosmin and MIB degradation. The carriers were colonized by mainly proteobacteria (80% relative abundance), with betaproteobacteria as the most prevalent sub-class (40% relative abundance). The latter taxon is known to harbor many nitrate-reducing bacterial species, as well as terpenes-degrading bacteria. Differential abundance analysis revealed a positive correlation between the abundance of the Thauera genera, a bacterial taxon specialized in terpenes (including geosmin and MIB) degradation under denitrifying conditions.

### Example 5: Effect of the Water Flow Mode on the Ability of the Oil-Hydrocolloid Carriers to Colonize TOCs-Degrading Bacteria

The ability of the oil-hydrocolloid carriers to form or colonize geosmin- and MIB-degrading bacteria when using different water flow modes was assessed by operating a upflow reactor (column) and a moving bed (MB) reactor. In the moving bed mode, water flows in a circular fashion within a spherical vessel. The water is circulated by an immersed pump. In the upflow mode, water is flown from the bottom of a 1 L column by a peristaltic pump. The water used in this experiment was taken from an aquaculture system. CFU (colony-forming units; cells/ml) was calculated by counting visible bacterial colonies formed on a petri dish inoculated with 0.1 ml of dissolved carriers (0.1 ml) and incubated for 48 hours under controlled environment. The number of colonies has then been multiplied by a factor of 10 to account for 1 ml.)

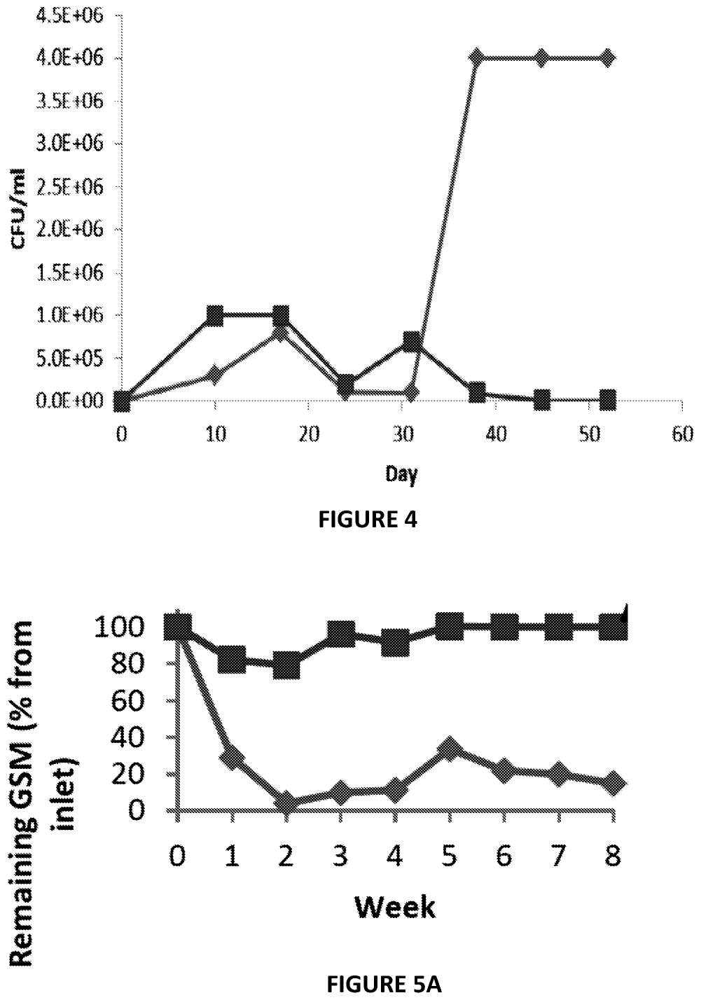

It was found that a larger bacteria population was obtained when using the moving bed reactor (FIG. 4). Without wishing to being bound by theory or mechanism of action, it is contemplated that the major difference in the bacteria growth conditions provided by the two operating modes is the concentration of dissolved oxygen. In the MB mode, high oxygen concentrations are maintained, while in the column upflow mode oxygen concentration is diminished to negligible levels. Accordingly, when the bacteria population size needs to be maintained within specific limits, a column reactor is preferable, in particular, wherein the water is circulated in an upflow fashion.

### Example 6: Long-Term Geosmin and MIB Removal by Columns Packed with Oil-Hydrocolloid Carriers

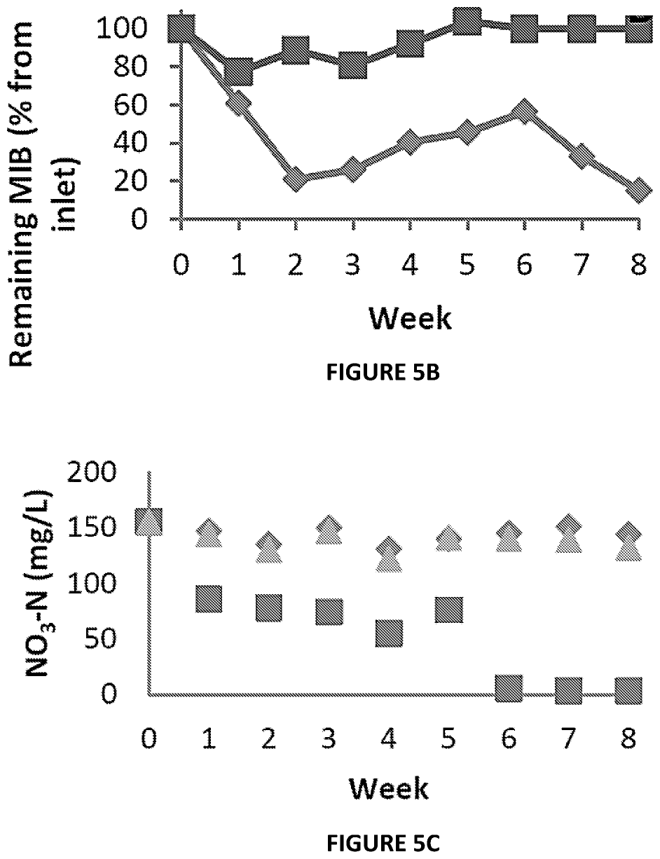

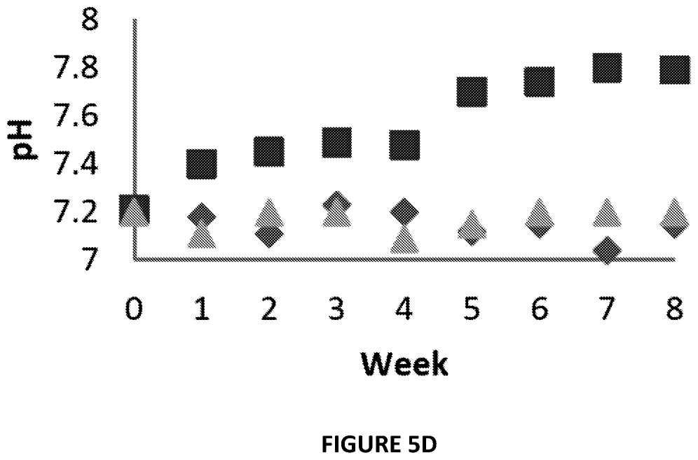

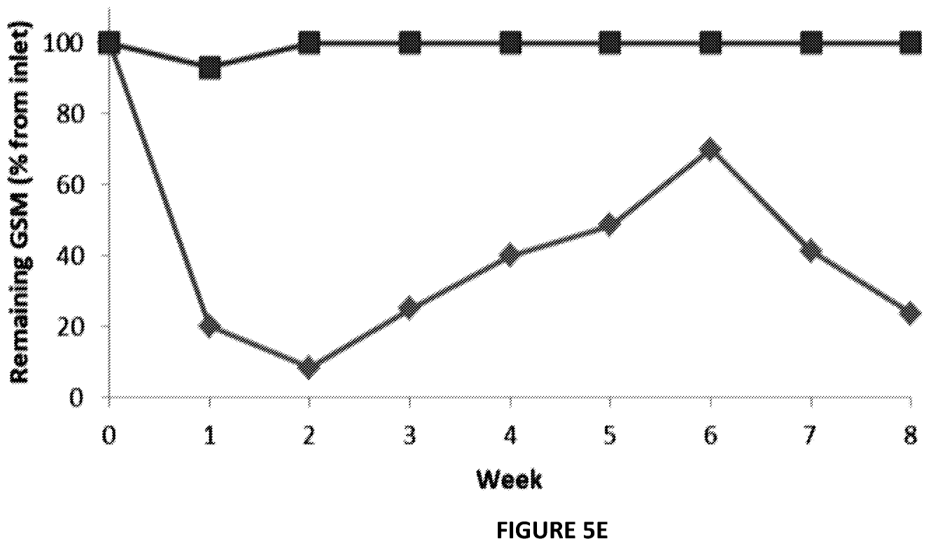

The long-term activity of the oil-hydrocolloid carriers was determined by operation of upflow reactors (columns) having a 1 L volume and containing 50 grams of 30% oil-containing carriers. The reactors were operated at a hydraulic retention time (HRT) of 5 hours and 10 hours and were fed with crude aquaculture water supplemented with 1000 ng/L of geosmin and MIB (each). Two additional columns, stocked with standard alginate carriers (without oil), which were operated at the same hydraulic retention times, served as controls. During operation of these systems for 8 weeks at 10 h HRT, it was found that:
(a) Hydrocolloid carriers containing 30% oil were capable of long-term removal of geosmin and MIB as compared to hydrocolloid carriers without oil (FIGS. 5A and 5B).
(b) Bacterial decomposition of geosmin and MIB underlies the observed geosmin and MIB removal as the total removal of these compounds exceeded by far the adsorption capacity of the carriers.
(c) In addition to geosmin and MIB removal, carriers were found to remove nitrate from the water (FIG. 5C). Without wishing to being bound by theory or mechanism of action, the removal of nitrate could be explained by the activity of denitrifiers as supported by pH values in the effluent of the columns, which were significantly higher than those in the influent (FIG. 5D).
(d) Operating the column at 10 h HRT resulted in more efficient geosmin removal than operation at 5 h HRT (FIG. 5E). However, operation at 10 h HRT also decreased nitrate concentration to minute amounts, which is undesirable in aquaculture systems, as it may result in formation of toxic and ecologically hazardous compounds, such as, e.g., sulfide. Operating the column at 5 h HRT resulted in a lesser decrease in nitrate concentration.

### Example 7: Wax-Oil-Hydrocolloid Carriers

Removal of geosmin by carriers comprising wax in addition to oil and hydrocolloid was tested. The tested carriers had the following compositions:
Alginate solution (1%), 30% beeswax;
64% Beeswax, 30% Sunflower Oil, 5% Alginate powder;
69% Beeswax, 30% Sunflower Oil, 1% Alginate powder; and
64% Beeswax, 30% Sunflower Oil, 1% Alginate powder, 5% Bentonite powder.

The wax-containing carriers were prepared as follows:

The wax was heated on a hot plate until melting. The hydrocolloid powder was then added to the molten wax and the hot mixture was poured into a mold and left there to solidify. After solidification, the carriers were removed from the mold, stored in containers and refrigerated until further use.

The wax and oil-containing carriers were prepared as follows:

The oil was heated on a hot plate until a temperature of 70-80° C. was reached. The wax was added gradually to the oil. After the wax was completely dissolved, the emulsifier was added (if needed). Then, the alginate solution or the alginate powder and bentonite were added to the mixture. The mixture was mixed on the heated stirrer until a homogeneous mixture was obtained.

The hot homogeneous mixture was then poured into molds and cooled at room temperature until solidification was achieved. After solidification, the carriers were removed from the mold, stored in containers and refrigerated until further use.

The carriers (3.5 g/L) were incubated in sterile DDW or sterile RAS water supplemented with geosmin (1000 ng/L). Incubation was carried out in an orbital shaker at room temp and 90 rpm for 24 hours. Concentration of geosmin in solutions were determined (in duplicates) at time=0 and after 24 hours of incubation. Bottles without carriers were used as controls. Geosmin removal efficiency by the carriers (also as compared to carriers of Example 1) is summarized in Table 1.

TABLE 1
Removal efficiency of geosmin by wax-containing carriers.

| | DDW | | RAS water | |
|---|---|---|---|---|
| | Removal | | Removal | |
| | efficiency | SD | efficiency | SD |
| Carrier type | (%) | (%) | (%) | (%) |
| Alginate solution (1%) + 30% | 70.10 | 3.50 | 64.30 | 2.70 |
| Sunflower oil + 1% Tween 80 | | | | |
| Alginate + Wax(30%) | 58.46 | 1.37 | 55.18 | 0.73 |
| Wax(64%) + Oil(30%) + | 74.28 | 3.77 | 63.48 | 0.56 |
| Alginate(5%) | | | | |
| Wax(69%) + Oil(30%) + | 75.05 | 4.76 | 76.21 | 3.82 |
| Alginate(1%) | | | | |
| Wax(64%) + Oil(30%) + | 81.97 | 1.44 | 79.56 | 1.21 |
| Alginate(1%) + Bentonite(5%) | | | | |
| Control (no carriers) | 3.00 | 0.50 | 2.11 | 1.32 |

Removal efficiency refers to the % of geosmin that was removed from the solutions during incubation.
Standard deviation (SD) is expressed as %.

Removal of MIB and geosmin from RAS water by three up-flow columns (1.3 L) packed with 50 g (wet weight) of wax-based carriers was further tested and compared with the carrier of Example 1. The tested carriers included:
69% Alginate solution (1%), 30% Sunflower oil, 1% Tween 80.
69% Beeswax, 30% Sunflower Oil, 1% Alginate powder.
64% Beeswax, 30% Sunflower Oil, 1% Alginate powder, 5% Bentonite powder.

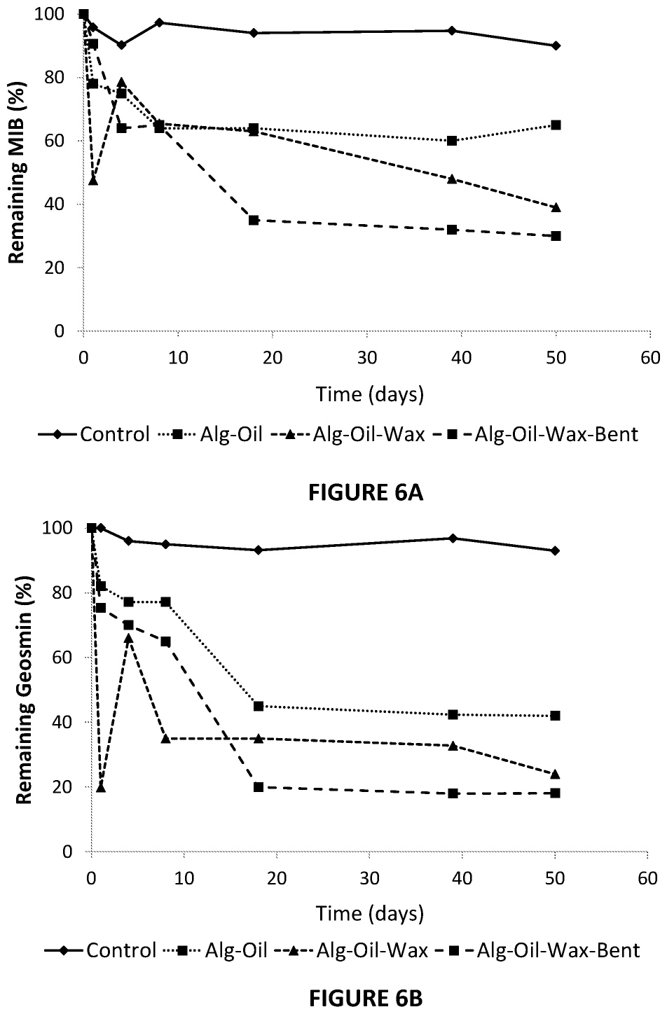

Each column was fed with water derived from an aquaculture recirculating system, by means of peristaltic pump, amended with 1000 ng/L of geosmin and MIB, each. Hydraulic retention time (HTR) was 4.3 hours. An additional column was operated under similar conditions, but without carriers, and served as control. The results of the up-flow column experiment are presents in FIGS. 6A-6B. Each value represents the mean of duplicate samples. Standard deviation was below 5%.

While the present invention has been particularly described, persons skilled in the art will appreciate that many variations and modifications can be made. Therefore, the invention is not to be construed as restricted to the particularly described embodiments, and the scope and concept of the invention will be more readily understood by reference to the claims, which follow.

1. A method for the removal of noxious taste or odor compounds (TOCs) from water of an aquaculture system, the method comprising contacting the water with a carrier comprising a hydrophobic agent configured to adsorb said noxious TOCs; and a hydrocolloid, wherein the carrier is adapted for the colonization of microorganisms which are capable of degrading said TOCs and wherein said TOCs comprise a terpenoid.

2. The method according to claim 1, wherein the hydrocolloid is configured to entrap the hydrophobic agent or wherein the hydrocolloid is dispersed within the hydrophobic agent.

3. (canceled)

4. The method according to claim 1, wherein the hydrophobic agent is selected from the group consisting of an oil, wax, fatty acid, fatty alcohol, and combinations thereof.

5. (canceled)

6. (canceled)

7. (canceled)

8. (canceled)

9. The method according to claim 1, wherein the hydrophobic agent comprises at least one of soybean oil, sunflower oil, and beeswax.

10. The method according to claim 1, wherein the hydrophobic agent is present in the carrier in a weight percent ranging from about 10% to about 40% out of the total wet weight of the carrier.

11. The method according to claim 10, wherein said carrier comprises soybean oil, which is present in a weight percent of about 30% out of the total wet weight of the carrier.

12. The method according to claim 1, wherein the hydrophobic agent is present in the carrier in a weight percent ranging from about 88% to about 99.5% out of the total dry weight of the carrier.

13. (canceled)

14. The method according to claim 13, wherein the hydrocolloid comprises alginate.

15. (canceled)

16. (canceled)

17. The method according to claim 1, wherein the carrier further comprises a cross-linking agent selected from the group consisting of calcium ion, magnesium ion, potassium ion, barium ion, aluminum ion, copper ion, lead ion, strontium ion, chitosan, poly(L-lysine), polyethyleneimine (PEI), and combinations thereof.

18. (canceled)

19. (canceled)

20. (canceled)

21. The method according to claim 1, wherein the carrier further comprises a filler selected from bentonite, kaolin, and any combination thereof.

22. The method according to claim 1, wherein the carrier comprises:
from about 10% (w/w) to about 40% (w/w) of the hydrophobic agent;
from about 0.1% (w/w) to about 5% (w/w) of the hydrocolloid;
from about 0.1% (w/w) to about 5% (w/w) of the cross-linking agent;
from about 0.001% (w/w) to about 1% (w/w) of the emulsifier; and
from about 50% (w/w) to about 95% (w/w) water,
out of the total wet weight of the carrier.

23. The method according to claim 22, wherein the hydrophobic agent comprises soybean oil, the hydrocolloid comprises alginate and the cross-linking agent comprises calcium ions.

24. (canceled)

25. (canceled)

26. (canceled)

27. The method according to any claim 1, wherein the carrier comprises:
from about 88% (w/w) to about 99.5% (w/w) of the hydrophobic agent;
from about 0.1% (w/w) to about 2% (w/w) of the hydrocolloid; and
from about 0.1% (w/w) to about 10% (w/w) of the filler,
out of the total dry weight of the carrier.

28. (canceled)

29. The method according to claim 1, wherein said microorganisms are endemic to the aquaculture system.

30. (canceled)

31. (canceled)

32. (canceled)

33. (canceled)

34. (canceled)

35. (canceled)

36. A recirculating aquaculture system (RAS) for maintaining aquaculture species, comprising:
a reservoir holding water and aquaculture species;
at least one of a solids removing filter, mechanical filter, and biological filter; and
a plurality of carriers comprising a hydrophobic agent configured to adsorb noxious taste or order compounds (TOCs), and a hydrocolloid, wherein said carriers are adapted for the colonization of microorganisms which are capable of degrading or transforming said TOCs.

37. The RAS according to claim 36, further comprising a reactor in fluid flow connection with the reservoir, and means for directing flow of a portion of the water from the reservoir to the reactor, wherein the reactor comprises the plurality of carriers.

38. The RAS according to claim 36, wherein the hydrophobic agent is selected from the group consisting of an oil, wax, fatty acid, fatty alcohol, and combinations thereof.

39. (canceled)

40. The RAS according to claim 36, wherein the hydrophobic agent is present in the carrier in a weight percent ranging from about 10% to about 40% out of the total wet weight of the carrier or from about 88% to about 99.5% out of the total dry weight of the carrier.

41. (canceled)

42. (canceled)

43. (canceled)

44. (canceled)

45. The RAS according to claim 36, wherein the plurality of carriers further comprise an emulsifier, selected from the group consisting of ethoxylated sorbitan esters of fatty acids, succinylated monoglycerides, sucrose esters of fatty acids, sucroglycerides, polyglycerol esters of fatty acids, polyglycerol polyricinoleate, propane-1,2-diol esters of fatty acids, propylene glycol esters of fatty acids, lactylated fatty acid esters of glycerol and propane-1, thermally oxidized soya bean oil interacted with mono- and diglycerides of fatty acids, dioctyl sodium sulfosuccinate, sodium stearoyl-2-lactylate, calcium stearoyl-2-lactylate, stearyl tartrate, stearyl citrate, sodium stearoyl fumarate, calcium stearoyl fumarate, sodium lauryl sulfate, ethoxylated mono- and di-glycerides, methyl glucoside-coconut oil ester, propane-1,2-diol, sorbitan monostearate, sorbitan tristearate, sorbitan monolaurate, sorbitan monooleate, sorbitan monopalmitate, sorbitan trioleate, polyoxypropylene-polyoxyethylene polymers, partial polyglycerol esters of polycondensed fatty acids of castor oil, stigmasterol-rich plant sterols, and combinations thereof.

46. (canceled)

47. (canceled)

48. The RAS according to claim 37, wherein said reactor is selected from the group consisting of a packed bed reactor, fixed bed reactor, moving bed reactor, rotating bed reactor, and fluidized bed reactor.

49. (canceled)

50. (canceled)
